(19) United States
(12) Patent Application Publication
(10) Pub. No.: US 2023/0398072 A1
(43) Pub. Date: Dec. 14, 2023

(54) CONCENTRATE CONTAINING POORLY SOLUBLE DRUG AND EMULSION PREPARED THEREFROM

(71) Applicant: BEIJING DELIVERY PHARMACEUTICAL TECHNOLOGY CO., LTD, Beijing (CN)

(72) Inventors: Cuishuan WU, Beijing (CN); Xiaobo CHENG, Beijing (CN); Dan ZHANG, Beijing (CN); Jixiang HOU, Beijing (CN); Boce ZHAO, Beijing (CN); Qiang ZHANG, Beijing (CN)

(73) Assignee: BEIJING DELIVERY PHARMACEUTICAL TECHNOLOGY CO., LTD, Beijing (CN)

(21) Appl. No.: 18/263,150

(22) PCT Filed: Dec. 10, 2021
(86) PCT No.: PCT/CN2021/137170

§ 371 Date: Jul. 27, 2023

(30) Foreign Application Priority Data
Jan. 28, 2021 (CN) 202110122277.1

(51) Int. Cl. A61K 9/107; A61K 47/24; A61K 47/10; A61K 47/14; A61K 47/26; A61K 47/44; A61K 31/635; A61K 31/192; A61K 31/337; A61K 9/00

(52) U.S. Cl. CPC A61K 9/107; A61K 47/24; A61K 47/10; A61K 47/14; A61K 47/26; A61K 47/44; A61K 31/635; A61K 31/192; A61K 31/337; A61K 9/0019

(57) ABSTRACT

A concentrate, characterized in that the concentrate contains a poorly soluble drug and a self-emulsifying carrier. The selfemulsifying carrier consists of the following substances: a composite emulsifier, which consists of phospholipid and nonphospholipid emulsifiers; an oil, which is medium chain triglyceride; and a co-emulsifier, which is anhydrous ethanol. The phospholipid is selected from soybean phospholipid, egg yolk lecithin, and a mixture thereof. The concentrate can be used to prepare an intravenous injection emulsion.

### FIELD OF THE INVENTION

The invention belongs to the field of pharmaceutical preparation. Specifically, the invention relates to a liquid concentrate comprising a poorly soluble drug and a process for preparing the same, as well as an emulsion prepared from the liquid concentrate.

### BACKGROUND OF THE INVENTION

The administration, particularly administration by injection of poorly soluble drugs has been one of the difficulties and hotspots of research in the field of pharmaceutical preparation, mainly for the reason that many poorly soluble drugs cannot be prepared into injections due to the solubility problem. In view of the irreplaceable role of injections in clinic, increasing the solubility of poorly soluble drugs and thereby realizing injection administration of poorly soluble drugs have become one of the most active research directions in the field of pharmaceutical preparation.

Diverse strategies for increasing the solubility of poorly soluble drugs have emerged in the past half a century, including formulating them into oil-in-water drug-loaded emulsions, liposomes, micelles, cyclodextrin inclusions, nanosuspensions, and the like, each of which has advantages and limitations.

The drug-loaded emulsion is the most common and relatively mature preparation technology for industrial manufacture, is often used for solubilization of poorly soluble drugs for injection administration, and can exhibit high drug load. Examples of poorly soluble drugs that are marketed in form of emulsion for injection include diazepam, felbinacethyl, flurbiprofen axetil, alprostadil, dexamethasone palmate, vitamin K2, and propofol. Although drug-loaded emulsions can achieve the administration by injection of poorly soluble drugs, they have a number of deficiencies. Most drugs that can be made into an emulsion are generally liquids with high log P value or solids with low melting point, while poorly soluble drugs having a melting point higher than 200° C. are not suitable for formulating into an emulsion. The existing drug-loaded emulsions have extremely high requirements on formula, process, equipment and formulation system, so that only a few manufacturers can master relevant technologies. Moreover, the cost is high. The commercially available drug-loaded emulsions have problems in physical, chemical or microbial stability with the following main manifestations: layering, reduced drug content, increased impurities and ease of contamination; many emulsions, particularly large volume emulsion for injection are not resistant to elevated temperature sterilization; the storage and transportation of the existing drug-loaded emulsions for injection require cold chain with high cost; the existing commercially available drug-loaded emulsions are not resistant to freezing-thawing, and their package inserts clearly indicate “the emulsion cannot be frozen and should be discarded after freezing”, which brings difficulty to transportation. The drug-loaded emulsions of the poorly soluble drugs at least should ensure the physical and chemical stability for more than 12 months, which is difficult to realize for many poorly soluble drugs.

Liposome technology has also been used to solubilize poorly soluble drugs to achieve their administration by injection. Liposomes are vesicles formed by encapsulating a drug in a phospholipid bilayer, in which the poorly soluble drug is dissolved. Examples of the poorly soluble drugs that are marketed in the form of liposomes for injection are paclitaxel, daunorubicin, adriamycin, amphotericin B and cytarabine. Liposomes, as a solubilizing technique of poorly soluble drugs for administration by injection, have such limitations as high cost and complicated process for large-scale production; another disadvantage of liposomes is the low drug-load. In addition, liposomes have poor stability and often require lyophilization.

Micelle is also a common approach to increase the solubility of poorly soluble drugs and realize their administration by injection. Poorly soluble antitumor agents that are widely used in clinic such as paclitaxel, docetaxel and cabazitaxel are solubilized by forming micelles with surfactants such as polyoxyethylene castor oil (e.g., kolliphor ELP) and Tween 80. However, micellar solutions tend to be diluted by blood after entering the blood circulation. Meanwhile, because of the significant amount of surfactants, adverse effects such as hemolysis and allergy are often caused. These problems become limitation of the application of micelles as solubilizing technology to the poorly soluble drugs for injection.

Cyclodextrin inclusion is also one of the solubilizing technologies of poorly soluble drugs for administration by injection. There are several products for injection prepared by cyclodextrin inclusion in market, such as alprostadil, itraconazole, voriconazole, posaconazole, mitomycin, ziprasidone, and remdesivir. However, for some poorly soluble drugs, the cyclodextrin inclusions have lower drug load. In addition, only a few of kinds of cyclodextrins are available, and cyclodextrins have significant nephrotoxicity. These problems bring challenges for the development of cyclodextrin inclusion formulations of poorly soluble drugs.

Nanosuspension is a formulation in which drug nano-particles are dispersed in an aqueous solution containing a stabilizer. The nanosuspension can be prepared as a preparation having a high dose of a poorly soluble drug. This way is particularly suitable for poorly soluble drugs which have very low solubility in water or are insoluble in both water and oil. The nanosuspension does not need any solubilizers and thus can avoid the defects of other drug delivery systems such as low drug load and increased adverse effects due to addition of carriers. The commercially available products for injection which are manufactured by nanosuspension technology include azacitidine, betamethasone, cortisone acetate, triamcinolone acetonide, dexamethasone, methylprednisolone, medroxyprogesterone acetate, hydrocortisone and the like. However, the nanosuspension technology has the defects such as complicated preparation process, high cost, difficult control of the particle size, and increased particle size after long-term storage.

There is still a need for compositions, particularly injectable compositions, comprising poorly soluble drugs and having good stability.

In view of the background above, in order to look for a stable composition comprising a poorly soluble drug, the inventors studied different types of compositions comprising poorly soluble drugs by various techniques using different drugs such as celecoxib as the model of poorly soluble drugs. For example, the inventors prepared various liquid formulations comprising celecoxib, such as oil-in-water emulsion, liposome, long-circulating nanosuspension, and micelle. However, these formulations exhibited respective problems. For example, the oil-in-water emulsion itself is a heterogeneous system, and the problems of layering, drug precipitation, etc. occurred upon storage; in addition, the preparation of the drug-loaded emulsion required a high-pressure homogenizer, and set high requirements on the formula, process, equipment and formulation system so that the cost is too high; furthermore, studies showed that the oil-in-water emulsion of celecoxib had problem with the physical, chemical and microbial stability. The long-circulating nanosuspension prepared by polylactic-co-glycolic acid (PLGA) failed to meet the requirement of intravenous injection, and could only be used for intramuscular or subcutaneous injection, which limited the application to patients in emergency.

Eventually, the inventors surprisingly found through a large number of experiments that clinically applicable dose of poorly soluble drugs could be prepared into a stable composition by adopting a specific composite emulsifier, an oil and a co-emulsifier. The composition can realize good dissolution of poorly soluble drugs, is a uniform and transparent oily solution, and can be prepared using an extremely simple process. The composition is a liquid concentrate which can be diluted into emulsion using an aqueous vehicle prior to use, and the formed emulsion can be directly and conveniently administered to patients, and has excellent stability. The vehicle may be an aqueous vehicle suitable for injection (e.g., water for injection, 5% dextrose injection. 0.9% sodium chloride injection, etc.) or an aqueous vehicle suitable for oral administration (e.g., purified water, diluted ethanol, etc.). Moreover, the emulsion prepared from the composition according to the invention fully satisfies the requirements of intravenous injection, and can be administered by intravenous injection.

In the Chinese patent application CN10834845 IA, a propofol microemulsion is prepared by diluting a propofol concentrate, which comprises propofol, at least one surfactant, at least one solvent, at least one co-surfactant and at least one co-solvent. The concentrate is substantially free of water. More specifically, in Example 1 of the Chinese patent application CN108348451A, a propofol concentrate B6 was prepared, comprising propofol, Solutol HS-15, propylene glycol, MCT, PEG400, ethanol and lecithin. Compared with the concentrate B6, the liquid concentrate of the present disclosure comprises fewer kinds of self-emulsifying carriers, requires a simpler preparation process, is suitable for more kinds of poorly soluble drugs, and causes fewer adverse effects when propofol is comprised.

Therefore, the composition according to the invention not only has excellent stability, but also comprises less kinds of self-emulsifying carriers (thereby reducing the risk brought by the carriers in terms of safety), requires a simpler preparation process, and is suitable for more kinds of poorly soluble drugs. The emulsion prepared from the composition according to the invention can smoothly achieve the administration, particularly administration by injection of poorly soluble drugs, and meets the clinical requirements which have not been met currently.

### SUMMARY OF THE INVENTION

One object of the invention is to provide a liquid concentrate containing a poorly soluble drug, and a simple, environment-friendly and easily industrialized process for preparing the liquid concentrate. Another object of the invention is to provide an emulsion, which can smoothly achieve administration, particularly administration by injection of a poorly soluble drug.

In the first aspect, the invention provides a liquid concentrate comprising a poorly soluble drug, characterized in that said liquid concentrate comprises a poorly soluble drag and a self-emulsifying carrier consisting of:
(1) A composite emulsifier consisting of phospholipid and a non-phospholipid emulsifier;
(2) An oil, which is medium chain triglyceride; and
(3) A co-emulsifier, which is absolute ethanol,
wherein the phospholipid is selected from the group consisting of soybean phospholipid, egg yolk lecithin, and a mixture thereof, preferably is egg yolk lecithin.

The non-phospholipid emulsifier is preferably selected from the group consisting of polyoxyethylene castor oil (e.g., polyoxyethylene 35 castor oil, pure polyoxyethylene 35 castor oil), polyoxyethylene hydrogenated castor oil (e.g., polyoxyethylene 40 hydrogenated castor oil, polyoxyethylene 60 hydrogenated castor oil), polyethylene glycol 15-hydroxystearate, D-alpha-tocopheryl polyethylene glycol 1000 succinate (TPGS), polysorbates (e.g., polysorbate 20, 21, 40, 60, 61, 65, 80, 81, 85, 120, particularly polysorbate 80), and a mixture thereof. More preferably, the non-phospholipid emulsifier is selected from the group consisting of polyoxyethylene 40 hydrogenated castor oil, polyoxyethylene 35 castor oil, pure polyoxyethylene 35 castor oil, polyethylene glycol 15-hydroxystearate, polysorbate 80, and a mixture thereof.

In an embodiment of the first aspect, in the liquid concentrate comprising a poorly soluble drug according to the invention, when the weight of the poorly soluble drug, the composite emulsifier, the oil and the co-emulsifier is regarded as 100%, the poorly soluble drug accounts for 0.01%-20%, preferably 0.1%-15%, more preferably 0.1%-12%, such as 0.1%. 0.5%, 1%. 1.5%, 2%, 5%, 6%, 10%, 12% by weight; the phospholipid accounts for 0.5-10%, preferably 1-5%, such as 1%. 2%. 3%, 4%. 5% by weight; the non-phospholipid emulsifier accounts for 30-70%, preferably 30-60%, more preferably 30-50%, such as 30%, 31%, 32%, 33%, 39%, 40%. 43%, 44%, 46%, 47%. 48%. 49%, 50% by weight: the medium chain triglyceride accounts for 20%-50%, preferably 20%-40%, more preferably 23%-40%, such as 20%, 23%, 24%, 25%. 28%, 29%, 30%, 32%, 33%. 35%. 37%, 40% by weight; the absolute ethanol accounts for the balance.

The aforesaid liquid concentrate comprising a poorly soluble drug may only consist of: the poorly soluble drug: the composite emulsifier consisting of phospholipid and the non-phospholipid emulsifier; the oil which is medium chain triglyceride; and the co-emulsifier which is absolute ethanol.

Alternatively, the aforesaid liquid concentrate comprising a poorly soluble drug may further comprise a pH adjuster and/or an antioxidant. The pH adjuster can be one or more selected from citric acid, citrate (such as sodium citrate), maleic acid, tartaric acid, hydrochloric acid, sodium hydroxide, acetic acid, acetate (such as sodium acetate), phosphoric acid, and phosphate (such as sodium hydrogen phosphate, sodium dihydrogen phosphate, or sodium phosphate). The antioxidant may be one or more selected from α-tocopherol succinate, ascorbyl palmitate, butylated hydroxyanisole (BHA), and butylated hydroxytoluene (BHT).

In the second aspect, the invention provides a process for preparing the liquid concentrate comprising a poorly soluble drug, comprising the steps of mixing the poorly soluble drug, the phospholipid, the non-phospholipid emulsifier, the medium chain triglyceride as the oil and the absolute ethanol as the co-emulsifier in any order, stirring to be uniform, filtering, filling, and capping to seal.

In the third aspect, the invention provides an emulsion, which is prepared by diluting the aforesaid liquid concentrate comprising a poorly soluble drug with an aqueous vehicle. The emulsion has an average particle size of between 20 nm and 4000 nm, preferably between 20 nm and 1000 nm, more preferably between 20 nm and 500 nm, and even more preferably between 20 nm and 300 nm.

In the fourth aspect, the invention provides the use of the aforesaid liquid concentrate comprising a poorly soluble drug in the manufacture of an emulsion, in particular an emulsion for intravenous injection.

### BRIEF DESCRIPTION OF THE FIGURES

FIG. 1 shows the pharmacodynamic experimental results produced by the test product (i.e., celecoxib injection obtained by diluting the liquid concentrate 2 according to the invention with 5% glucose injection in a ratio of 1 g:100 ml), the positive controls (i.e., commercially available parecoxib sodium injection and flurbiprofen axetil injection), the negative control (i.e., 5% glucose injection), and the blank formulation (the formulation obtained by replacing the poorly soluble drug in the test product with the same weight of absolute ethanol) in the rat model of incisional pain of Example 7. The ordinate of FIG. 1 is the 50% withdrawal threshold (g), and the abscissa of FIG. 1 is time (hours). In FIG. 1, the first curve (▴) represents the experimental results of the test product, celecoxib injection; the second curve (▪) represents the experimental results of the positive control, flurbiprofen axetil injection; the third curve (●) represents the experimental results of the positive control, parecoxib sodium injection; the fourth curve (∘) represents the experimental results of the blank formulation; and the fifth curve (▾) represents the experimental results of the negative control. 5% glucose injection. In FIG. 1, *** represents that the test product, celecoxib emulsion injection has a significant difference compared with the parecoxib sodium injection (p<0.01), ## represents that the test product, celecoxib emulsion injection has a significant difference compared with the flurbiprofen axetil injection (p<0.05), and ### represents that the test product, celecoxib emulsion injection has a significant difference compared with the flurbiprofen axetil injection (p<0.01).

### DETAILED DESCRIPTION OF THE INVENTION

### Embodiments

Embodiment 1. A liquid concentrate, characterized in that, the liquid concentrate comprises a poorly soluble drug and a self-emulsifying carrier consisting of:
(1) A composite emulsifier consisting of phospholipid and a non-phospholipid emulsifier;
(2) An oil, which is medium chain triglyceride; and
(3) A co-emulsifier, which is absolute ethanol,
wherein the phospholipid is selected from the group consisting of soybean phospholipid, egg yolk lecithin, and a mixture thereof.

Embodiment 2. The liquid concentrate according to Embodiment 1, wherein the non-phospholipid emulsifier is selected from the group consisting of polyoxyethylene castor oil (e.g., polyoxyethylene 35 castor oil, pure polyoxyethylene 35 castor oil), polyoxyethylene hydrogenated castor oil (e.g., polyoxyethylene 40 hydrogenated castor oil, polyoxyethylene 60 hydrogenated castor oil), polyethylene glycol 15-hydroxystearate, D-alpha-tocopheryl polyethylene glycol 1000 succinate (TPGS), polysorbates (e.g., polysorbate 20, 21, 40, 60, 61, 65, 80, 81, 85, 120, particularly polysorbate 80), and a mixture thereof.

Embodiment 3. The liquid concentrate according to Embodiment 2, wherein the non-phospholipid emulsifier is selected from the group consisting of polyoxyethylene 40 hydrogenated castor oil, polyoxyethylene 35 castor oil, pure polyoxyethylene 35 castor oil, polyethylene glycol 15-hydroxystearate, polysorbate 80, and a mixture thereof.

Embodiment 4. The liquid concentrate according to any one of Embodiments 1 to 3, wherein when the weight of the poorly soluble drug, the composite emulsifier, the oil and the co-emulsifier is regarded as 100%, the poorly soluble drug accounts for 0.01% to 20% by weight, preferably 0.1% to 15% by weight, more preferably 0.1% to 12% by weight; the phospholipid accounts for 0.5% to 10% by weight, preferably 1% to 5% by weight; the non-phospholipid emulsifier accounts for 30% to 70% by weight, preferably 30% to 60% by weight, more preferably 30% to 50% by weight; and the medium chain triglyceride accounts for 20% to 50% by weight, preferably 20% to 40% by weight, more preferably 23% to 40% by weight; and the absolute ethanol accounts for the balance.

Embodiment 5. The liquid concentrate according to any one of Embodiments 1 to 4, wherein the poorly soluble drug is selected from the group consisting of celecoxib, valdecoxib, etocoxib, ibuprofen, dexibuprofen, propofol, flurbiprofen axetil, alprostadil, clevidipine butyrate, dexamethasone palmitate, felodipine, nimodipine, nifedipine, nitrendipine, cyclosporine, tacrolimus, levosimendan, adefovir dipivoxil, erythromycin, roxithromycin, posaconazole, itraconazole, voriconazole, miconazole, ketoconazole, progesterone, coenzyme Q10, clopidogrel, paclitaxel, docetaxel, cabazitaxel, etoposide, teniposide, hydroxycamptothecin, irinotecan, ubenimex, cisplatin, carboplatin, capecitabine, oxaliplatin, gefitinib, doxonthicin, vinblastine, vincristine, vinpocetine, vindesine, piroxicam, spironolactone, valproic acid, tamoxifen, azithromycin, vitamin A, vitamin D, vitamin E, vitamin K, fenofibrate, indomethacin, and remdesivir; preferably, the poorly soluble drug is selected from the group consisting of celecoxib, ibuprofen, dexibuprofen, propofol, flurbiprofen axetil, alprostadil, clevidipine butyrate, dexamethasone palmitate, felodipine, nimodipine, nifedipine, nitrendipine, cyclosporine, tacrolimus, levosimendan, adefovir dipivoxil, erythromycin, roxithromycin, posaconazole, itraconazole, voriconazole, progesterone, coenzyme Q10, clopidogrel, paclitaxel, docetaxel, cabazitaxel, etoposide, teniposide, clevidipine butyrate, remdesivir, carboplatin, valdecoxib, azithromycin, and etocoxib; most preferably, the poorly soluble drug is selected from the group consisting of celecoxib, paclitaxel, docetaxel, ibuprofen, nimodipine, coenzyme Q10, cabazitaxel, etocoxib, posaconazole, cyclosporine, flurbiprofen axetil, dexibuprofen, levosimendan, clevidipine butyrate, clopidogrel, remdesivir, tacrolimus, carboplatin, dexamethasone palmitate, valdecoxib, azithromycin, and propofol.

Embodiment 6. The liquid concentrate according to any one of Embodiments 1 to 5, further comprising a pH adjuster, an antioxidant, or the both.

Embodiment 7. The liquid concentrate according to any one of Embodiments 1 to 5, wherein
The poorly soluble drug is celecoxib, the phospholipid is egg yolk lecithin, the non-phospholipid emulsifier is polyethylene glycol 15-hydroxystearate, the oil is medium chain triglyceride, the co-emulsifier is absolute ethanol, and when the weight of the poorly soluble drug, the composite emulsifier, the oil and the co-emulsifier is regarded as 100%, celecoxib accounts for 5% by weight, the egg yolk lecithin accounts for 3% by weight, polyethylene glycol 15-hydroxystearate accounts for 48% by weight, the medium chain triglyceride accounts for 28% by weight, and the absolute ethanol accounts for 16% by weight;
or
The poorly soluble drug is ibuprofen, the phospholipid is egg yolk lecithin, the non-phospholipid emulsifier is polyethylene glycol 15-hydroxystearate, the oil is medium chain triglyceride, the co-emulsifier is absolute ethanol, the liquid concentrate further comprises citric acid, and when the weight of the poorly soluble drug, the composite emulsifier, the oil and the co-emulsifier is regarded as 100%, ibuprofen accounts for 12% by weight, the egg yolk lecithin accounts for 2% by weight, polyethylene glycol 15-hydroxystearate accounts for 48% by weight, the medium chain triglyceride accounts for 27% by weight, and the absolute ethanol accounts for 11% by weight;
or
The poorly soluble drug is docetaxel, the phospholipid is egg yolk lecithin, the non-phospholipid emulsifier is polyethylene glycol 15-hydroxystearate, the oil is medium chain triglyceride, the co-emulsifier is absolute ethanol, and when the weight of the poorly soluble drug, the composite emulsifier, the oil and the co-emulsifier is regarded as 100%, docetaxel accounts for 2% by weight, the egg yolk lecithin accounts for 3% by weight, the polyethylene glycol 15-hydroxystearate accounts for 48% by weight, the medium chain triglyceride accounts for 28% by weight, and the absolute ethanol accounts for 19% by weight;
or
The poorly soluble drug is paclitaxel, the phospholipid is egg yolk lecithin, the non-phospholipid emulsifier is polyethylene glycol 15-hydroxystearate, the oil is medium chain triglyceride, the co-emulsifier is absolute ethanol, the liquid concentrate further comprises citric acid, and when the weight of the poorly soluble drug, the composite emulsifier, the oil and the co-emulsifier is regarded as 100%, paclitaxel accounts for 1.5% by weight, the egg yolk lecithin accounts for 3% by weight, the polyethylene glycol 15-hydroxystearate accounts for 49.1% by weight, the medium chain triglyceride accounts for 29.4% by weight, and the absolute ethanol accounts for 17% by weight.

Embodiment 8. Use of the liquid concentrate according to any one of Embodiments 1 to 7 in the manufacture of an emulsion, in particular an emulsion for intravenous injection, for example intravenous infusion.

Embodiment 9. The use according to Embodiment 8, wherein the emulsion has an average particle size of between 20 nm and 4000 nm, preferably between 20 nm and 1000 nm, more preferably between 20 nm and 500 nm, and even more preferably between 20 nm and 300 nm.

Embodiment 10. A process for preparing the liquid concentrate according to any one of Embodiments 1 to 7, comprising the steps of mixing the poorly soluble drug, the phospholipid, the non-phospholipid emulsifier, the medium chain triglyceride and the absolute ethanol in any order, stirring to be uniform, filtering, filling, and capping to seal.

Embodiment 11. An emulsion obtained by diluting the liquid concentrate according to any one of Embodiments 1 to 7 with an aqueous vehicle.

Embodiment 12. The emulsion according to Embodiment 11, wherein the aqueous vehicle is an aqueous vehicle suitable for injection selected from the group consisting of water for injection, 5% dextrose injection, and 0.9% sodium chloride injection.

Embodiment 13. The emulsion according to Embodiment 12, wherein the emulsion is used for intravenous injection, in particular intravenous infusion.

### Composition of the Invention and the Process for Preparing the Same

The inventors found through research on the basic physicochemical properties of poorly soluble drugs that a stable composition can be prepared from a poorly soluble drug such as celecoxib using a composite emulsifier consisting of phospholipid and a non-phospholipid emulsifier, and a specific co-emulsifier and an oil. The composition is a liquid concentrate which form an emulsion upon dilution with an aqueous vehicle prior to use, for example, with an aqueous vehicle suitable for injection such as water for injection, 5% glucose injection, or 0.9% sodium chloride injection. The emulsion can be directly administered by injection, for example, intravenous injection, particularly intravenous infusion.

Accordingly, in the first aspect, the invention provides a liquid concentrate comprising a poorly soluble drug, characterized in that said liquid concentrate comprises: a poorly soluble drug: a composite emulsifier consisting of phospholipid and a non-phospholipid emulsifier; an oil, which is medium chain triglyceride; and a co-emulsifier, which is absolute ethanol.

The phospholipid is selected from the group consisting of soybean phospholipid, egg yolk lecithin, and a mixture thereof, preferably is egg yolk lecithin.

The non-phospholipid emulsifier is preferably selected from the group consisting of polyoxyethylene castor oil (e.g., polyoxyethylene 35 castor oil, pure polyoxyethylene 35 castor oil), polyoxyethylene hydrogenated castor oil (e.g., polyoxyethylene 40 hydrogenated castor oil, polyoxyethylene 60 hydrogenated castor oil), polyethylene glycol 15-hydroxystearate, D-alpha-tocopheryl polyethylene glycol 1000 succinate (TPGS), polysorbates (e.g., polysorbate 20, 21, 40, 60, 61, 65, 80, 81, 85, 120, particularly polysorbate 80), and a mixture thereof. More preferably, the non-phospholipid emulsifier is selected from the group consisting of polyoxyethylene 40 hydrogenated castor oil, polyoxyethylene 35 castor oil, pure polyoxyethylene 35 castor oil, polyethylene glycol 15-hydroxystearate, polysorbate 80, and a mixture thereof.

In a preferred embodiment of the first aspect, in the liquid concentrate comprising a poorly soluble drug according to the invention, when the weight of the poorly soluble drug, the composite emulsifier, the oil and the co-emulsifier is regarded as 100%, the poorly soluble drug accounts for 0.01% to 20%, preferably 0.1% to 15%, more preferably 0.1% to 12%, such as 0.1%, 0.5%, 1%, 1.5%, 2%, 5%, 6%, 10%, 12% by weight; the phospholipid accounts for 0.5% to 10%, preferably 1% to 5%, such as 1%, 2%, 3%. 4%, 5% by weight; the non-phospholipid emulsifier accounts for 30% to 70%, preferably 30% to 60%, more preferably 30% to 50%, such as 30%, 31%, 32%, 33%, 39%. 40%, 43%. 44%, 46%, 47%, 48%, 49%, 50% by weight; the medium chain triglyceride accounts for 20% to 50%, preferably 20% to 40%, more preferably 23% to 40%, such as 20%, 23%, 24%, 25%, 28%, 29%, 30%, 32%. 33%. 35%, 37%, 40% by weight; the absolute ethanol accounts for the balance.

The liquid concentrate comprising a poorly soluble drug according to the invention is a uniform and transparent oily solution and has good physical and chemical stability. The liquid concentrate remained as a uniform and transparent oily solution without layering and drug precipitation after storage at room temperature for 6 months. For example, the chemical stability of the liquid concentrates prepared in Example 4 of the present disclosure were investigated at 40±2° C. and 60±5% RH, and the results showed that the liquid concentrates did not show any significant changes in the content of the poorly soluble drugs and the relevant substances after storage at 40±2° C. and 60±5% RH for 6 months, and satisfied the requirements of the drug quality control standard.

The liquid concentrate comprising a poorly soluble drug according to the invention can be diluted with an aqueous vehicle to form an emulsion. The aqueous vehicle may be an aqueous vehicle suitable for injection (e.g., water for injection. 5% dextrose injection. 0.9% sodium chloride injection, etc.) or an aqueous vehicle suitable for oral administration (e.g., purified water, diluted ethanol, etc.). The emulsion formed by diluting the liquid concentrate comprising a poorly soluble drug according to the invention with an aqueous solvent has an average particle diameter of 20 nm to 4000 nm, preferably 20 nm to 1000 nm, more preferably 20 nm to 500 nm, and still more preferably 20 nm to 300 nm. The emulsion formed by diluting the liquid concentrate comprising a poorly soluble drug according to the invention with an aqueous solvent suitable for injection such as 5% dextrose injection has an average particle diameter of not more than 4000 nm, and meets the requirements for intravenous injection and even intravenous infusion. Thus, the emulsion can be used for subcutaneous injection, intradermal injection, intraperitoneal injection, and for intravenous injection, including intravenous bolus and intravenous infusion.

The liquid concentrate according to the invention itself has good stability, and the emulsion obtained by dilution of the liquid concentrate also has good stability. For example, when the emulsion obtained by diluting the liquid concentrate according to the invention, specifically the liquid concentrate 1 with 5% glucose injection in a ratio of 1 g:100 ml, is left at room temperature for 24 hours, no drug precipitation or layering occurred, and the pH value kept stable in the range of 4.5 to 7.0.

In addition, the liquid concentrate according to the invention has several additional advantages. Specifically, compared with the concentrate disclosed in the Chinese patent application CN108348451A, the composition according to the invention comprises fewer kinds of self-emulsifying carriers, requires a simpler preparation process, is suitable for more kinds of poorly soluble drugs, and has fewer adverse effects.

The term “poorly soluble drug(s)” as used herein refers to a drug which is known to be applicable in the medicinal field and has low solubility in water relative to the effective dosing amount thereof. More specifically, the “poorly soluble drug” described herein refers to a drug which is “slightly soluble” (solute 1 g (ml) can be dissolved in 100 to less than 1,000 ml of solvent), “very slightly soluble” (solute 1 g (ml) can be dissolved in 1,000 to less than 10,000 ml of solvent), or “practically insoluble or insoluble” (solute 1 g (ml) cannot be completely dissolved in 10,000 ml of solvent), according to the “General Notices” of the Chinese Pharmacopoeia.

Examples of the “poorly soluble drug(s)” described herein include, but are not limited to celecoxib, valdecoxib, etocoxib, ibuprofen, dexibuprofen, propofol, flurbiprofen axetil, alprostadil, clevidipine butyrate, dexamethasone palmitate, felodipine, nimodipine, nifedipine, nitrendipine, cyclosporine, tacrolimus, levosimendan, adefovir dipivoxil, erythromycin, roxithromycin, posaconazole, itraconazole, voriconazole, miconazole, ketoconazole, progesterone, coenzyme Q10, clopidogrel, paclitaxel, docetaxel, cabazitaxel, etoposide, teniposide, hydroxycamptothecin, irinotecan, ubenimex, cisplatin, carboplatin, capecitabine, oxaliplatin, gefitinib, doxorubicin, vinblastine, vincristine, vinpocetine, vindesine, piroxicam, spironolactone, valproic acid, tamoxifen, azithromycin, vitamin A, vitamin D, vitamin E, vitamin K, fenofibrate, indomethacin, remdesivir. Preferably, the poorly soluble drug is selected from the group consisting of celecoxib, ibuprofen, dexibuprofen, propofol, flurbiprofen axetil, alprostadil, clevidipine butyrate, dexamethasone palmitate, felodipine, nimodipine, nifedipine, nitrendipine, cyclosporine, tacrolimus, levosimendan, adefovir dipivoxil, erythromycin, roxithromycin, posaconazole, itraconazole, voriconazole, progesterone, coenzyme Q10, clopidogrel, paclitaxel, docetaxel, cabazitaxel, etoposide, teniposide, clevidipine butyrate, remdesivir, carboplatin, valdecoxib, azithromycin and etocoxib. Most preferably, the poorly soluble drug is selected from the group consisting of celecoxib, paclitaxel, docetaxel, ibuprofen, nimodipine, coenzyme Q10, cabazitaxel, etocoxib, posaconazole, cyclosporine, flurbiprofen axetil, dexibuprofen, levosimendan, clevidipine butyrate, clopidogrel, remdesivir, tacrolimus, carboplatin, dexamethasone palmitate, valdecoxib, azithromycin and propofol.

The inventors investigated the oils that may be used in the liquid concentrate according to the invention, and the experimental results showed that the best stability was obtained using medium chain triglyceride. If medium chain trigyceride is replaced by soybean oil for injection, olive oil, fish oil, corn oil, structural oil, castor oil, sunflower seed oil, cottonseed oil, tea oil, etc., the obtained composition showed layering, non-uniform system and poor formulation formability.

The inventors screened the emulsifiers that may be used in the liquid concentrate according to the invention, and the investigated phospholipid emulsifiers included soybean phospholipid, egg yolk lecithin and hydrogenated soybean lecithin; the investigated non-phospholipid emulsifiers included polyoxyethylene 40 hydrogenated castor oil (e.g. kolliphor RH40), polyoxyethylene 35 castor oil (e.g. kolliphor EL and kolliphor ELP), polyethylene glycol 15-hydroxystearate (e.g. kolliphor HS15), D-alpha-tocopheryl polyethylene glycol 1000 succinate (TPGS), polysorbate 80 (e.g. Tween 80). Experimental results showed that the emulsions formed by diluting the compositions prepared using phospholipid or non-phospholipid emulsifier alone had poor stability, and layered after about 2-4 hours; only when both the phospholipid and non-phospholipid emulsifiers were used in combination, the resulting liquid concentrates were stable and uniform, and the emulsions obtained after dilution of the liquid concentrates had good stability and met all the requirements of preparations for intravenous injection.

The inventors also investigated co-emulsifiers, and the experimental results showed that the best stability was obtained when absolute ethanol was used as co-emulsifier. If the ethanol was replaced by propylene glycol, glycerin, PEG200, PEG300. PEG400, etc., the resulting composition was not uniform, exhibited layering, and had poor formulation formability.

Most preferably, the liquid concentrates according to the invention are those having the components and proportions indicated in Example 4 of the present disclosure.

The aforesaid liquid concentrate comprising a poorly soluble drug according to the invention may only consist of: the poorly soluble drug; the composite emulsifier consisting of phospholipid and the non-phospholipid emulsifier; the oil which is medium chain trigyceride; and the co-emulsifier which is absolute ethanol.

Alternatively, the aforesaid liquid concentrate comprising a poorly soluble drug according to the invention may further comprise additional components such as a pH adjuster and/or an antioxidant. The pH adjuster can be one or more selected from citric acid, citrate (such as sodium citrate), maleic acid, tartaric acid, hydrochloric acid, sodium hydroxide, acetic acid, acetate (such as sodium acetate), phosphoric acid, and phosphate (such as sodium hydrogen phosphate, sodium dihydrogen phosphate, or sodium phosphate). The antioxidant may be one or more selected from α-tocopherol succinate, ascorbyl palmitate, butylated hydroxyanisole (BHA), and butylated hydroxytoluene (BHT).

In the second aspect, the invention provides a process for preparing the liquid concentrate comprising a poorly soluble drug, characterized in that the process comprises the steps of mixing the poorly soluble drug, the phospholipid, the non-phospholipid emulsifier, the medium chain triglyceride and the absolute ethanol in any order, stirring to be uniform, filtering, filling, and capping to seal.

The inventors found that the process for preparing the liquid concentrate according to the invention is very simple, and the order of addition of the individual components, the stirring time, and the like have no influence on the quality of the liquid concentrate, provided that the dissolution of the poorly soluble drug is ensured.

The invention successfully prepared the liquid concentrate comprising a poorly soluble drug using a very simple process, which has good stability and can be diluted with an aqueous vehicle to give an emulsion which is directly used for administration by intravenous injection. Compared with the prior art, the liquid concentrate according to the invention has simplified formula, and the preparation process thereof is simple, can be achieved by simple mixing, stirring, filtering, filling and capping, does not require a homogenizer, a microfluidizer or a complicated formulation system, and can be performed by an ordinary enterprise. Moreover, the liquid concentrate according to the invention has significantly improved physical and chemical stability; the storage and transportation thereof do not require cold-chain, so that the cost of manufacture, transportation, storage and use is largely reduced, and great convenience is provided for clinical medication.

In the third aspect, the invention provides an emulsion, which is prepared by diluting the aforesaid liquid concentrate comprising a poorly soluble drug with an aqueous vehicle. The emulsion has an average particle size of between 20 nm and 4000 nm preferably between 20 nm and 1000 nm, more preferably between 20 nm and 500 nm, and even more preferably between 20 nm and 300 nm.

The emulsion has good stability within 24 hours.

The emulsion can be administered to a patient for the treatment of a disease that may be treated by the poorly soluble drug comprised therein. For example, the emulsion comprising celecoxib can be used in the treatment of acute pain and inflammatory diseases such as osteoarthritis and rheumatoid arthritis: the emulsion comprising paclitaxel or docetaxel can be used in the treatment of cancers, for example, solid tumors such as breast cancer, ovarian cancer, head and neck cancer, lung cancer (including non-small cell lung cancer and small cell lung cancer), pancreatic cancer, gastric cancer, melanoma, soft tissue sarcoma; the emulsion comprising ibuprofen or dexibuprofen can be used in the treatment of pain, such as headache, joint pain, migraine, dental pain, muscular pain, neuralgia, dysmenorrhea. The poorly soluble drugs and their uses described herein are known in the art, and the prior art literatures describing the use of these poorly soluble drugs are considered a part of the present disclosure.

In the fourth aspect, the invention provides the use of the aforesaid liquid concentrate comprising a poorly soluble drug in the manufacture of an emulsion. The emulsion is particularly useful for intravenous injection, for example intravenous bolus and intravenous infusion.

In the present disclosure, the inventors thoroughly studied the factors that influence the formation and the physical and chemical stability of the liquid concentrate comprising a poorly soluble drug, as well as the physical stability and efficacy of the emulsion obtained by dilution of the liquid concentrate, using various drugs such as celecoxib as the model of a poorly soluble drug, and obtained the liquid concentrate according to the invention. The liquid concentrate can be widely applied to poorly soluble drugs, realize the injection administration of poorly soluble drugs, and provide new possible treatment for clinical application.

### Definitions

Throughout the present disclosure, the terms “composition comprising a poorly soluble drug”, “liquid concentrate comprising a poorly soluble drug”, “composition according to the invention”. “liquid concentrate according to the invention” are used interchangeably and refer to a composition comprising a poorly soluble drug, a composite emulsifier consisting of phospholipid and a non-phospholipid emulsifier, medium chain triglyceride as an oil and absolute ethanol as a co-emulsifier, unless the context indicates otherwise.

The term “self-emulsifying carrier” as used herein refers to a pharmaceutically acceptable carrier which facilitates the formation of emulsion upon dilution of the liquid concentrate according to the invention with an aqueous vehicle.

The term “consisting of” or “consist of” as used herein means that no substance other than the specified components is comprised in a significant amount. For example, in the liquid concentrate according to the invention, “the self-emulsifying carrier consisting of a composite emulsifier, an oil and a co-emulsifier” means that no significant amount of other substances that facilitate the formation of emulsion are comprised in addition to the composite emulsifier, the oil and the co-emulsifier specified herein.

The term “medium chain trigyceride” as used herein refers to the non-volatile vegetable oil extracted from the hard dried endosperm of coconut or the dried endosperm of oil palm, which is a mixture of saturated fatty acid triglycerides. Medium chain trigyceride is commercially available, for example, from Shinsun Pharma, China and IOI Oleo GmbH, Germany.

The term “absolute ethanol” as used herein refers to ethanol having a purity of up to 99.5% or more.

The term “polyoxyethylene castor oil” as used herein refers to the substance obtained by reacting varying amounts of ethylene oxide and castor oil. Examples of polyoxyethylene castor oil include, but are not limited to, polyoxyethylene 35 castor oil, and pure polyoxyethylene 35 castor oil.

The term “polyoxyethylene 35 castor oil” as used herein refers to the substance obtained by reacting 1 mol of glycerol ricinoleate with 35 mol of ethylene oxide, which contains a small amount of polyethylene glycol ricinoleate and free ethylene glycol in addition to polyoxyethylene glycerol triricinoleate. Polyoxyethylene 35 castor oil is commercially available, for example from BASF under the tradenames kolliphor EL and kolliphor ELP.

The term “pure polyoxyethylene 35 castor oil” as used herein refers to purified polyoxyethylene glycerol triricinoleate, which is substantially free of polyethylene glycol ricinoleate and free ethylene glycol.

The term “polyoxyethylene hydrogenated castor oil” as used herein refers to the substance obtained by reacting varying amounts of ethylene oxide and hydrogenated castor oil. Examples of polyoxyethylene hydrogenated castor oil include, but are not limited to, polyoxyethylene 40 hydrogenated castor oil and polyoxyethylene 60 hydrogenated castor oil.

The term “polyoxyethylene 40 hydrogenated castor oil” as used herein refers to the substance obtained by reacting 1 mol of glycerol trihydroxystearate with 40-45 mol of ethylene oxide, which contains a small amount of polyethylene glycol trihydroxystearate and free polyethylene glycol, in addition to polyoxyethylene glycerol trihydroxystearate. Polyoxyethylene 40 hydrogenated castor oil is commercially available, for example from BASF under the tradename kolliphor RH 40.

The term “polyoxyethylene 60 hydrogenated castor oil” as used herein refers to the substance obtained by reacting 1 mol of glycerol trihydroxystearate with 60 mol of ethylene oxide, which contains a small amount of polyethylene glycol trihydroxystearate and free polyethylene glycol, in addition to polyoxyethylene glycerol trihydroxystearate.

The term “polyethylene glycol 15-hydroxystearate” as used herein is commercially available, for example, from BASF or Sigma-Aldrich under the tradename kolliphor HS15 or Solutol HS-15.

The term “D-alpha-tocopheryl polyethylene glycol 1000 succinate (TPGS)” as used herein is a water-soluble derivative of vitamin E, which is obtained by reacting the carboxyl group of vitamin E succinate with the hydroxyl group of polyethylene glycol. TPGS is commercially available, for example, from BASF under the tradename Tocophersolan (TPGS).

The term “polysorbate” as used herein refers to a series of partial fatty acid esters of polyoxyethylene sorbitan, which is obtained by copolymerization of 1 mole of sorbitol and about 20.5 or 4 moles of ethylene oxide. Examples of polysorbates include, but are not limited to, for example, polysorbate 20, 21, 40, 60, 61, 65, 80, 81, 85, 120, and in particular polysorbate 80. Polysorbates are commercially available, for example from Nanjing Well Pharmaceutical Co., LTD, under the tradenames Tween 20, Tween 40, Tween 80 and the like.

The term “about”, “approximately”, or “around” as used herein means that the numerical values given thereafter can be expanded by ±20%. For example, “about 100%” means 80% to 120%.

The term “substantially free of” as used herein means that the content of the substance thereafter is less than 1%.

### EXAMPLES

The following examples are intended to illustrate the invention, but not to limit in any way the scope thereof as defined by the appended claims.

Unless specified otherwise, the particle size and polydispersity index (PDI) were measured using a laser particle sizer (NicompZ3000, PSS. USA), the pH values were measured using a pH meter (PB-10, Sartorius), high performance liquid chromatography was performed using Shimadzu LC-20AT, the temperatures were given in degrees Celsius, and the operations without specific temperature were performed at ambient temperature.

The meanings of abbreviations used in the Examples are shown in Table 1.

TABLE 1
the meanings of abbreviations

| Abbre- | | |
|---|---|---|
| viation | Meaning | Manufacturer |
| ELP | Polyoxyethylene 35 castor oil kolliphor EL | BASF |
| EPC | Egg yolk lecithin, EPC 80 | Lipoid |
| SPC | Soybean phospholipid | Jiangsu Maxim |
| | | Biological Co., |
| | | Ltd. |
| HSPC | Hydrogenated soybean lecithin | Lipoid |
| RH40 | Polyoxyethylene 40 hydrogenated castor oil, | BASF |
| | kolliphor RH40 | |
| HS15 | Polyethylene glycol 15-hydroxystearate, | BASF |
| | kolliphor HS15 | |
| Tween | Tween 80 | Nanjing Well |
| 80 | | Pharmaceutical |
| | | Co., Ltd. |
| TPGS | D-alpha-tocopheryl polyethylene glycol | BASF |
| | 1000 succinate | |
| MCT | medium chain triglyceride | Shinsun Pharma |
| LCT | Soybean oil | Shinsun Pharma |

### Example 1: Screening of Emulsifiers

### 1. Formulas 1-5: Screening of Single Emulsifier

TABLE 2
Formulas comprising single emulsifier

| Component | Formula 1 | Formula 2 | Formula 3 | Formula 4 | Formula 5 |
|---|---|---|---|---|---|
| Celecoxib | 0.5 g | 0.5 g | 0.5 g | 0.5 g | 0.5 g |
| MCT | 3.7 g | 3.7 g | 3.7 g | 3.7 g | 3.7 g |
| HS15 | 3.9 g | — | — | — | — |
| Tween 80 | — | 3.9 g | — | — | — |
| ELP | — | — | 3.9 g | — | — |
| TPGS | — | — | — | 3.9 g | — |
| RH40 | — | — | — | — | 3.9 g |
| Absolute | 1.9 g | 1.9 g | 1.9 g | 1.9 g | 1.9 g |
| ethanol | | | | | |
| In total | 10 g | 10 g | 10 g | 10 g | 10 g |

Process: individual components in the amount specified for each of the formulas above were added to a 20 ml vial and magnetically stirred until all components were completely dissolved.

The liquid concentrates obtained by Formulas 1-5 were all uniform and transparent oily solutions. The resulting oily solutions were diluted with 5% glucose injection in a ratio of 1 g:100 ml. After standing at room temperature, the formation of emulsion was observed and the stability of the resulting emulsion was examined. The results were shown in Table 3.

TABLE 3
Stability of emulsions formed by dilution of liquid
concentrates prepared using a single emulsifier

| Item | Formula 1 | Formula 2 | Formula 3 | Formula 4 | Formula 5 |
|---|---|---|---|---|---|
| Appearance | Uniform and | Uniform and | Uniform and | Uniform and | Uniform and |
| of liquid | transparent | transparent | transparent | transparent | transparent |
| concentrate | oily solution | oily solution | oily solution | oily solution | oily solution |
| Formation of | White | White | White | White | White |
| emulsion | emulsion | emulsion | emulsion | emulsion | emulsion |
| | formed | formed | formed | formed | formed |
| Stability of | Layering in | Layering in | Layering in | Layering in | Layering in |
| emulsion | around 2 h | around 1 h | around 2 h | around 2 h | around 2 h |

The above experimental data showed that all the liquid concentrates prepared using HS15, Tween 80, ELP, TPGS or RH40 as single emulsifier were uniform and transparent oily solutions and formed white emulsions upon dilution with 5% glucose injection, but the formed emulsions quickly layered, indicating poor emulsion stability.

Since none of the liquid concentrates prepared using a single emulsifier formed a stable emulsion upon dilution, the particle size of these emulsions was not determined.

### 2. Formulas 6-11: Screening of Composite Emulsifier

TABLE 4
Formulas comprising composite emulsifier

| | Formula | Formula | Formula | Formula | Formula | Formula |
|---|---|---|---|---|---|---|
| Component | 6 | 7 | 8 | 9 | 10 | 11 |
| Celecoxib | 0.5 g | 0.5 g | 0.5 g | 0.5 g | 0.5 g | 0.5 g |
| MCT | 3.7 g | 3.7 g | 3.7 g | 3.7 g | 4.0 g | 3.7 g |
| HS15 | 3.9 g | 3.9 g | 3.9 g | — | 1.9 g | — |
| Tween 80 | — | — | — | — | 2.0 g | — |
| EPC | 0.3 | — | — | 0.3 | — | 2.0 g |
| SPC | — | 0.3 g | — | 3.9 | — | — |
| HSPC | — | — | 0.3 g | — | — | 1.9 g |
| Absolute | 1.6 g | 1.6 g | 1.6 g | 1.6 g | 1.6 g | 1.6 g |
| ethanol | | | | | | |
| In total | 10 g | 10 g | 10 g | 10 g | 10 g | 10 g |

Process: individual components in the amount specified for each of the formulas above were added to a 20 ml vial and magnetically stirred until all components were completely dissolved.

Experimental results showed that Formulas 8, 9 and 11 failed to form a uniform and transparent oily solution; Formulas 6, 7 and 10 formed uniform and transparent oily solutions, and the emulsions formed from these oily solutions did not exhibit layering after standing.

All the liquid concentrates prepared by Formulas 6-11 formed white emulsions upon dilution with 5% glucose injection in a ratio of 1 g:100 ml. The emulsions were left at room temperature for 24 hours before the measurement of particle size using a laser particle sizer (NicompZ3000, PSS, USA). The results were shown in Table 5.

TABLE 5
Particle size and stability of emulsions formed by diluting the liquid concentrates
prepared using a composite emulsifier

| | 0 h | | 2 h | | 4 h | | 8 h | | 24 h | |
|---|---|---|---|---|---|---|---|---|---|---|
| | MD | PDI | MD | PDI | MD | PDI | MD | PDI | MD | PDI |
| Formula 6 | 100.6 | 0.273 | 102.4 | 0.223 | 98.5 | 0.226 | 108.6 | 0.216 | 101.5 | 0.262 |
| Formula 7 | 112.6 | 0.314 | 114.1 | 0.579 | 104.0 | 0.556 | 117.2 | 0.518 | 110.7 | 0.598 |
| Formula 8 | 1254.2 | 0.767 | — | — | — | — | — | — | — | — |
| Formula 9 | 1315.5 | 0.627 | — | — | — | — | — | — | — | — |
| Formula 10 | 332.6 | 0.385 | 389.2 | 0.531 | — | — | — | — | — | — |
| Formula 11 | 2316.6 | 0.724 | — | — | — | — | — | — | — | — |

“—” represents that the particle size was not measured due to the occurrence of layering;
“MD” means the average particle size (average diameter) in nm;
“PDI” represents the particle size dispersion index (Polydispersity index).

As shown in Table 5, after the emulsions above were left at room temperature for 24 hours, the particle size of the emulsions formed by the oily solutions prepared by Formula 6 comprising the composite emulsifier consisting of HS15 and EPC and by Formula 7 comprising the composite emulsifier consisting of HS15 and SPC did not significantly change; the other emulsions had large and unstable particle size, and layered in about 2 hours after the emulsions were formed so that the emulsions could not satisfy the requirements of administration by intravenous injection.

Therefore, a formula comprising a composite emulsifier consisting of phospholipid and a non-phospholipid emulsifier should be used and said phospholipid should not be HSPC.

### Example 2: Screening of Oil

The inventors investigated various pharmaceutically acceptable oils, and the specific experimental designs were as follows:

TABLE 6
Formulas comprising different oils

| | Formula | Formula | Formula | Formula | Formula | Formula | Formula | Formula | Formula | Formula |
|---|---|---|---|---|---|---|---|---|---|---|
| Component | 12 | 13 | 14 | 15 | 16 | 17 | 18 | 19 | 20 | 21 |
| Celecoxib | 0.5 g | 0.5 g | 0.5 g | 0.5 g | 0.5 g | 0.5 g | 0.5 g | 0.5 g | 0.5 g | 0.5 g |
| HS15 | 5.0 g | 5.0 g | 5.0 g | 5.0 g | 5.0 g | 5.0 g | 5.0 g | 5.0 g | 5.0 g | 5.0 g |
| EPC | 0.3 g | 0.3 g | 0.3 g | 0.3 g | 0.3 g | 0.3 g | 0.3 g | 0.3 g | 0.3 g | 0.3 g |
| MCT | 2.6 g | | | | | | | | | |
| LCT | | 2.6 g | | | | | | | | |
| Olive oil | | | 2.6 g | | | | | | | |
| Fish oil | | | | 2.6 g | | | | | | |
| Com oil | | | | | 2.8 g | | | | | |
| Structural oil | | | | | | 2.6 g | | | | |
| Castor oil | | | | | | | 2.6 g | | | |
| Sunflower oil | | | | | | | | 2.6 g | | |
| Cottonseed oil | | | | | | | | | 2.6 g | |
| Tea oil | | | | | | | | | | 2.6 g |
| Absolute ethanol | 1.6 g | 1.6 g | 1.6 g | 1.6 g | 1.6 g | 1.6 g | 1.6 g | 1.6 g | 1.6 g | 1.6 g |
| In total | 10 g | 10 g | 10 g | 10 g | 10 g | 10 g | 10 g | 10 g | 10 g | 10 g |

Process: individual components in the amount specified for each of the formulas above were added to a 20 ml vial and magnetically stirred until all components were completely dissolved.

The appearance of the liquid concentrates prepared by Formulas 12-21 was observed. The liquid concentrates prepared by Formulas 12-21 were diluted with 5% glucose injection in a ratio of 1 g:100 ml, and the formed emulsions were left at room temperature for 24 hours. The formation of the emulsions from the liquid concentrates was observed, and the particle size of the resulting emulsions was measured and the stability thereof was examined. The results were shown in Table 7.

TABLE 7
Liquid concentrates prepared by Formulas 12-21 and stability of emulsions formed upon deletion thereof

| | | Formula | Formula | Formula | Formula | Formula | Formula | Formula | Formula | Formula |
|---|---|---|---|---|---|---|---|---|---|---|
| | Formula 12 | 13 | 14 | 15 | 16 | 17 | 18 | 19 | 20 | 21 |
| Appearance of | Uniform and | Turbid | Turbid | Turbid | Turbid | Turbid | Turbid | Turbid | Turbid | Turbid |
| liquid | transparent | liquid, | liquid, | liquid, | liquid, | Liquid, | liquid, | liquid, | liquid, | liquid, |
| concentrate | oily solution | layering | layering | layering | layering | layering | layering | layering | layering | layering |
| | | after | after | after | after | after | after | after | after | after |
| | | standing | standing | standing | standing | standing | standing | standing | standing | standing |
| | | for 1 h | for 1 h | for 1 h | for 1 h | for 1 h | for 1 h | for 1 h | for 1 h | for 1 h |
| Formation of | Emulsion | — | — | — | — | — | — | — | — | — |
| emulsion upon | with obvious | | | | | | | | | |
| dilution | slightly blue | | | | | | | | | |
| | opalescence | | | | | | | | | |
| Stability of | Stable after | — | — | — | — | — | — | — | — | — |
| emulsion | standing for | | | | | | | | | |
| | 24 h | | | | | | | | | |
| Average | 30 nm | — | — | — | — | — | — | — | — | — |
| particle size of | | | | | | | | | | |
| emulsion (nm) | | | | | | | | | | |

“—”: The liquid concentrates were turbid and layered after standing at room temperature for 1 hour, showing that the liquid concentrates were not stable systems. Thus, no further investigation was made.

As can be seen from the experimental results in Table 7, only the liquid concentrate prepared by Formula 12 comprising medium chain triglyceride was uniform and transparent oily solutions, and formed stable emulsion upon dilution. The liquid concentrates prepared by Formulas 13-21 comprising such oils for injection as soybean oil, olive oil, fish oil, corn oil, structural oil, castor oil, sunflower seed oil, cottonseed oil, and tea oil were turbid, failed to form uniform and transparent oily solutions, and layered after landing at room temperature for around 1 hour. Therefore, MCT should be used as the oil.

### Example 3: Screening of Co-Emulsifier in Liquid Concentrate Comprising Celecoxib

The inventors investigated various pharmaceutically acceptable co-emulsifiers, and the specific experimental design was as follows:

TABLE 8
Formulas comprising different co-emulsifiers

| Component | Formula 22 | Formula 23 | Formula 24 | Formula 25 | Formula 26 | Formula 27 |
|---|---|---|---|---|---|---|
| Celecoxib | 0.5 g | 0.5 g | 0.5 g | 0.5 g | 0.5 g | 0.5 g |
| HS15 | 3.0 g | 3.0 g | 3.0 g | 3.0 g | 3.0 g | 3.0 g |
| EPC | 0.3 g | 0.3 g | 0.3 g | 0.3 g | 0.3 g | 0.3 g |
| MCT | 4.0 g | 4.0 g | 4.0 g | 4.0 g | 4.0 g | 4.0 g |
| Absolute | 1.6 g | | | | | |
| ethanol | | | | | | |
| Propylene | | 1.6 g | | | | |
| glycol | | | | | | |
| Glycerol | | | 1.6 g | | | |
| PEG200 | | | | 1.6 g | | |
| PEG300 | | | | | 1.6 g | |
| PEG400 | | | | | | 1.6 g |
| In total | 10 g | 10 g | 10 g | 10 g | 10 g | 10 g |

Process: individual components in the amount specified for each of the formulas above were added to a 20 ml vial and magnetically stirred until all components were completely dissolved.

The appearance of the liquid concentrates prepared by Formulas 22-27 was observed. The liquid concentrates prepared by Formulas 22-27 were diluted with 5% glucose injection in a ratio of 1 g:100 ml, and the formed emulsions were left at room temperature for 24 hours. The formation of the emulsions from the liquid concentrates was observed, and the particle size of the resulting emulsions was measured and the stability thereof was examined. The results were shown in Table 9.

TABLE 9
Liquid concentrates prepared by Formulas 22-27 and stability of
emulsions formed upon deletion thereof

| | Formula | Formula | Formula | Formula | Formula | Formula |
|---|---|---|---|---|---|---|
| | 22 | 23 | 24 | 25 | 26 | 27 |
| Appearance | Uniform | Turbid | Turbid | Turbid | Turbid | Turbid |
| of liquid | clear oily | liquid, | liquid, | liquid, | liquid, | liquid. |
| concentrate | solution | layering | layering | layering | layering | layering |
| | | after | after | after | after | after |
| | | standing | standing | standing | standing | standing |
| | | for 1 h | for 1 h | for 1 h | for 1 h | for 1 h |
| Formation | Emulsion | — | — | — | — | — |
| of | with | | | | | |
| emulsion | obvious | | | | | |
| upon | slightly | | | | | |
| dilution | blue | | | | | |
| | opalescence | | | | | |
| Stability | Stable after | — | — | — | — | — |
| of | standing for | | | | | |
| emulsion | 24 h | | | | | |
| Average | 230 nm | — | — | — | — | — |
| particle | | | | | | |
| size | | | | | | |
| of | | | | | | |
| emulsion | | | | | | |
| (nm) | | | | | | |

“—”: The liquid concentrates were turbid and layered after standing at room temperature for 1 hour, showing that the liquid concentrates were not stable systems. Thus, no further investigation was made.

As can be seen from the experimental results in Table 9, only the liquid concentrate prepared by Formula 22 comprising absolute ethanol was uniform and transparent oily solution, and formed stable emulsion upon dilution. The liquid concentrates prepared by Formulas 23-27 comprising such co-emulsifiers as propylene glycol, glycerol. PEG200, PEG300, PEG400 were turbid, and layered after standing at room temperature for around 1 hour. Therefore, absolute ethanol should be used as the co-emulsifier.

### Example 4: Preparation of Liquid Concentrates Comprising Poorly Soluble Drugs and the Study on the Particle Size and Stability of Emulsions Prepared Therefrom (*the Total Weight of the Poorly Soluble Drug, the Composite Emulsifier, the Oil and the Co-Emulsifier is Regarded as 100%)

### Liquid Concentrate 1

Formula:

| Component | % by weight * | Amount | |
|---|---|---|---|
| Celecoxib | 5% | 0.5 | g |
| MCT | 28% | 2.8 | g |
| HS15 | 48% | 4.8 | g |
| EPC | 2.8% | 0.28 | g |
| Absolute ethanol | 16.2% | 1.62 | g |
| In total | | 10 | g |

Process:

EPC and celecoxib in the amount specified for the formula above were weighed into a 20 ml vial. Absolute ethanol in the amount specified for the formula above was added followed by stirring at 2000 rpm in a water bath at 60° C. for 1 min. MCT and HS15 were added in the amount specified for the formula above followed by stirring at 2000 rpm in a water bath at 60° C. for 5 min to give a uniform and transparent oily solution.

Study on Stability of Emulsion:

Liquid concentrate 1 was diluted with 5% glucose injection in a ratio of 1 g:100 ml. The particle size and stability of the resulting emulsion were examined for 24 hours.

| Item | 0 h | 24 h |
|---|---|---|
| Appearance | Emulsion with slightly | Emulsion with slightly |
| | blue opalescence | blue opalescence, no |
| | | drug precipitation |
| Average particle | 30.70 | 29.62 |
| size (nm) | | |
| PDI | 0.02 | 0.01 |

### Liquid Concentrate 2

Formula:

| Component | % by weight * | Amount | |
|---|---|---|---|
| Celecoxib | 5% | 0.5 | g |
| MCT | 28% | 2.8 | g |
| HS15 | 48% | 4.8 | g |
| EPC | 3% | 0.3 | g |
| Absolute ethanol | 16% | 1.59 | g |
| Citric acid | | 0.01 | g |
| In total | | 10 | g |

Process:

EPC in the amount specified for the formula above was weighed into a 20 ml vial. Absolute ethanol in the amount specified for the formula above was added followed by stirring at 2000 rpm in a water bath at 60° C. for 1 min to dissolve EPC. Celecoxib in the amount specified for the formula above was added followed by stirring at 2000 rpm in a water bath at 60° C. for 1 min. HS15. MCT and citric acid in the amount specified for the formula above were added followed by stirring at 2000 rpm in a water bath at 60° C. for 5 min to give a uniform and transparent oily solution.

Study on Stability of Emulsion:

Liquid concentrate 2 was diluted with 5% glucose injection in a ratio of 1 g:100 ml. The particle size and stability of the resulting emulsion were examined for 24 hours.

| Item | 0 h | 24 h |
|---|---|---|
| Appearance | Transparent emulsion | Transparent emulsion with |
| | with slightly blue | slightly blue opalescence, |
| | opalescence | no drug precipitation |
| Average particle | 30.05 | 28.16 |
| size (nm) | | |
| PDI | 0.01 | 0.02 |

### Liquid Concentrate 3

Formula:

| Component | % by weight * | Amount |
|---|---|---|
| Paclitaxel | 2% | 0.2 g |
| MCT | 40% | 4.0 g |
| HS15 | 33% | 3.3 g |
| EPC | 5% | 0.5 g |
| Absolute ethanol | 20% | 2.0 g |
| In total | | 10 g |

Process:

EPC in the amount specified for the formula above was weighed into a 20 ml vial. Absolute ethanol in the amount specified for the formula above was added followed by stirring at 2000 rpm in a water bath at 60° C. for 1 min to dissolve EPC. Paclitaxel in the amount specified for the formula above was added followed by stirring at 2000 rpm in a water bath at 60° C. for 1 min. HS15 and MCT in the amount specified for the formula above were added followed by stirring at 2000 rpm in a water bath at 60° C. for 5 min to give a uniform and transparent oily solution.

Study on Stability of Emulsion:

Liquid concentrate 3 was diluted with 5% glucose injection in a ratio of 1 g:50 ml. The particle size and stability of the resulting emulsion were examined for 24 hours.

| Item | 0 h | 24 h |
|---|---|---|
| Appearance | White emulsion | White emulsion, no drug |
| | | precipitation |
| Average particle size (nm) | 255.34 | 257.46 |
| PDI | 0.21 | 0.25 |

### Liquid Concentrate 4

Formula:

| Component | % by weight * | Amount |
|---|---|---|
| docetaxel | 2% | 0.2 g |
| MCT | 28% | 2.8 g |
| HS15 | 48% | 4.8 g |
| EPC | 3% | 0.3 g |
| Absolute ethanol | 19% | 1.9 g |
| In total | | 10 g |

Process:

EPC, docetaxel, absolute ethanol, MCT and HS15 in the amount specified for the formula above were weighed into a 20 ml vial followed by stirring at 2000 rpm in a water bath at 60° C. for 5 min to give a uniform and transparent oily solution.

Study on Stability of Emulsion:

Liquid concentrate 4 was diluted with 5% glucose injection in a ratio of 1 g:50 ml. The particle size and stability of the resulting emulsion were examined for 24 hours.

| Item | 0 h | 24 h |
|---|---|---|
| Appearance | Emulsion with slightly | Emulsion with slightly |
| | blue opalescence | blue opalescence, no |
| | | drug precipitation |
| Average particle | 50.21 | 49.47 |
| size (nm) | | |
| PDI | 0.16 | 0.20 |

### Liquid Concentrate 5

Formula:

| Component | % by weight * | Amount |
|---|---|---|
| Ibuprofen | 10% | 1.0 g |
| MCT | 23% | 2.3 g |
| HS15 | 48% | 4.8 g |
| EPC | 3% | 0.3 g |
| Absolute ethanol | 16% | 1.6 g |
| In total | | 10 g |

Process:

EPC, ibuprofen, absolute ethanol, MCT and HS15 in the amount specified for the formula above were weighed into a 20 ml vial followed by stirring at 2000 rpm in a water bath at 60° C. for to give a uniform and transparent oily solution.

Study on Stability of Emulsion:

Liquid concentrate 5 was diluted with 5% glucose injection in a ratio of 1 g:100 ml. The particle size and stability of the resulting emulsion were examined for 24 hours.

| Item | 0 h | 24 h |
|---|---|---|
| Appearance | Emulsion with | Emulsion with obvious |
| | obvious slightly blue | slightly blue opalescence, |
| | opalescence | no drug precipitation |
| Average particle | 36.35 | 37.25 |
| size (nm) | | |
| PDI | 0.11 | 0.15 |

### Liquid Concentrate 6

Formula:

| Component | % by weight * | Amount | |
|---|---|---|---|
| Nimodipine | 0.1% | 0.01 | g |
| MCT | 37% | 3.7 | g |
| HS15 | 43.9% | 4.39 | g |
| EPC | 3% | 0.3 | g |
| Absolute ethanol | 16% | 1.6 | g |
| In total | | 10 | g |

Process:

EPC, nimodipine, absolute ethanol. MCT and HS15 in the amount specified for the formula above were weighed into a 20 ml vial followed by stirring at 2000 rpm in a water bath at 60° C. for 5 min to give a uniform and transparent oily solution.

Study on Stability of Emulsion:

Liquid concentrate 6 was diluted with 5% glucose injection in a ratio of 1 g:50 ml. The particle size and stability of the resulting emulsion were examined for 24 hours.

| Item | 0 h | 24 h |
|---|---|---|
| Appearance | Slightly yellow | Slightly yellow emulsion, no |
| | emulsion | drug precipitation |
| Average particle | 166.37 | 167.28 |
| size (nm) | | |
| PDI | 0.19 | 0.21 |

### Liquid Concentrate 7

Formula:

| Component | % by weight * | Amount |
|---|---|---|
| Coenzyme Q10 | 5% | 0.5 g |
| MCT | 28% | 2.8 g |
| TPGS | 48% | 4.8 g |
| EPC | 1% | 0.1 g |
| Absolute ethanol | 18% | 1.8 g |
| In total | | 10 g |

Process:

EPC, coenzyme Q10, absolute ethanol, MCT and TPGS in the amount specified for the formula above were weighed into a 20 ml vial followed by stirring at 2000 rpm in a water bath at 60° C. for 5 min to give a uniform and transparent oily solution.

Study on Stability of Emulsion:

Liquid concentrate 7 was diluted with 5% glucose injection in a ratio of 1 g:100 ml. The particle size and stability of the resulting emulsion were examined for 24 hours.

| Item | 0 h | 24 h |
|---|---|---|
| Appearance | Transparent yellow | Transparent yellow emulsion, no |
| | emulsion | drug precipitation |
| Average particle | 30.13 | 31.64 |
| size (nm) | | |
| PDI | 0.06 | 0.05 |

### Liquid Concentrate 8

Formula:

| Component | % by weight * | Amount |
|---|---|---|
| Cabazitaxel | 2% | 0.2 g |
| MCT | 32% | 3.2 g |
| HS15 | 47% | 4.7 g |
| EPC | 5% | 0.5 g |
| Absolute ethanol | 14% | 1.4 g |
| In total | | 10 g |

Process:

EPC, cabazitaxel, absolute ethanol, MCT and HS15 in the amount specified for the formula above were weighed in order into a 20 ml vial followed by stirring at 2000 rpm in a water bath at 60° C. for 5 min to give a uniform and transparent oily solution.

Study on Stability of Emulsion:

Liquid concentrate 8 was diluted with 5% glucose injection in a ratio of 1 g:50 ml. The particle size and stability of the resulting emulsion were examined for 24 hours.

| Item | 0 h | 24 h |
|---|---|---|
| Appearance | White emulsion | White emulsion, no drug |
| | | precipitation |
| Average particle size (nm) | 30.13 | 31.64 |
| PDI | 0.06 | 0.05 |

### Liquid Concentrate 9

Formula:

| Component | % by weight * | Amount |
|---|---|---|
| Etocoxib | 5% | 0.5 g |
| MCT | 30% | 3.0 g |
| Tween 80 | 46% | 4.6 g |
| SPC | 3% | 0.3 g |
| Absolute ethanol | 16% | 1.6 g |
| In total | | 10 g |

Process:

SPC and etocoxib in the amount specified for the formula above were weighed into a 20 ml vial. The absolute ethanol in the amount specified for the formula above was added followed by stirring at 2000 rpm in a water bath at 60° C. for 1 min. Then, MCT and Tween 80 in the amount specified for the formula above were added followed by stirring at 2000 rpm in a water bath at 60° C. for 5 min to give a uniform and transparent oily solution.

Study on Stability of Emulsion:

Liquid concentrate 9 was diluted with 5% glucose injection in a ratio of 1 g:100 ml. The particle size and stability of the resulting emulsion were examined for 24 hours.

| Item | 0 h | 24 h |
|---|---|---|
| Appearance | White emulsion | White emulsion, no drug |
| | | precipitation |
| Average particle size (nm) | 126.12 | 130.14 |
| PDI | 0.14 | 0.15 |

### Liquid Concentrate 10

Formula:

| Component | % by weight * | Amount |
|---|---|---|
| Posaconazole | 1% | 0.1 g |
| MCT | 25% | 2.5 g |
| RH40 | 54% | 5.4 g |
| SPC | 3% | 0.3 g |
| Absolute ethanol | 17% | 1.7 g |
| In total | | 10 g |

Process:

SPC in the amount specified for the formula above was weighed into a 20 ml vial. Absolute ethanol in the amount specified for the formula above was added followed by stirring at 2000 rpm in a water bath at 60° C. for 1 min to dissolve EPC. Posaconazole in the amount specified for the formula above was added followed by stirring at 2000 rpm in a water bath at 60° C. for 1 min. RH40 and MCT in the amount specified for the formula above were added followed by stirring at 2000 rpm in a water bath at 60° C. for 5 min to give a uniform and transparent oily solution.

Study on Stability of Emulsion:

Liquid concentrate 10 was diluted with 5% glucose injection in a ratio of 1 g:50 ml. The particle size and stability of the resulting emulsion were examined for 12 hours.

| Item | 0 h | 12 h |
|---|---|---|
| Appearance | Emulsion with blue | Emulsion with blue opalescence, |
| | opalescence | no drug precipitation |
| Average particle | 105.56 | 107.33 |
| size (nm) | | |
| PDI | 0.15 | 0.17 |

### Liquid Concentrate 11

Formula:

| Component | % by weight * | Amount |
|---|---|---|
| Cyclosporine | 2% | 0.2 g |
| MCT | 33% | 3.3 g |
| ELP | 40% | 4.0 g |
| EPC | 5% | 0.5 g |
| Absolute ethanol | 20% | 2.0 g |
| In total | | 10 g |

Process:

EPC in the amount specified for the formula above was weighed into a 20 ml vial. Absolute ethanol in the amount specified for the formula above was added followed by stirring at 2000 rpm in a water bath at 60° C. for linin to dissolve EPC. Cyclosporine in the amount specified for the formula above was added followed by stirring at 2000 rpm in a water bath at 60° C. for 1 min. ELP and MCT in the amount specified for the formula above were added followed by stirring at 2000 rpm in a water bath at 60° C. for 5 min to give a uniform and transparent oily solution.

Study on Stability of Emulsion:

Liquid concentrate 11 was diluted with 5% glucose injection in a ratio of 1 g:50 ml. The particle size and stability of the resulting emulsion were examined for 24 hours.

| Item | 0 h | 24 h |
|---|---|---|
| Appearance | Emulsion with blue | Emulsion with blue opalescence, |
| | opalescence | no drug precipitation |
| Average particle | 124.41 | 127.65 |
| size (nm) | | |
| PDI | 0.18 | 0.20 |

### Liquid Concentrate 12

Formula:

| Component | % by weight * | Amount |
|---|---|---|
| Flurbiprofen axetil | 1% | 0.1 g |
| MCT | 28% | 2.8 g |
| HS15 | 49% | 4.9 g |
| EPC | 3% | 0.3 g |
| Absolute ethanol | 19% | 1.9 g |
| In total | | 10 g |

Process:

EPC, flurbiprofen axetil, absolute ethanol, MCT and HS15 in the amount specified for the formula above were weighed in order into a 20 ml vial followed by stirring at 2000 rpm in a water bath at 60° C. for 5 min to give a uniform and transparent oily solution.

Study on Stability of Emulsion:

Liquid concentrate 12 was diluted with 5% glucose injection in a ratio of 1 g:50 ml. The particle size and stability of the resulting emulsion were examined for 24 hours.

| Item | 0 h | 24 h |
|---|---|---|
| Appearance | Clear emulsion | Clear emulsion, no drug |
| | | precipitation |
| Average particle size (nm) | 28.15 | 27.37 |
| PDI | 0.08 | 0.09 |

### Liquid Concentrate 13

Formula:

| Component | % by weight * | Amount |
|---|---|---|
| Dexibuprofen | 10% | 1.0 g |
| MCT | 23% | 2.3 g |
| ELP | 48% | 4.8 g |
| SPC | 3% | 0.3 g |
| Absolute ethanol | 16% | 1.6 g |
| In total | | 10 g |

Process:

SPC, dexibuprofen, absolute ethanol, MCT and ELP in the amount specified for the formula above were weighed in order into a 20 ml vial followed by stirring at 2000 rpm in a water bath at 60° C. for 5 min to give a uniform and transparent oily solution.

Study on Stability of Emulsion:

Liquid concentrate 13 was diluted with 5% glucose injection in a ratio of 1 g:100 ml. The particle size and stability of the resulting emulsion were examined for 24 hours.

| Item | 0 h | 24 h |
|---|---|---|
| Appearance | Emulsion with | Emulsion with obvious |
| | obvious slightly blue | slightly blue opalescence, |
| | opalescence | no drug precipitation |
| Average particle | 30.58 | 31.37 |
| size (nm) | | |
| PDI | 0.15 | 0.18 |

### Liquid Concentrate 14

Formula:

| Component | % by weight * | Amount | |
|---|---|---|---|
| Levosimendan | 0.1% | 0.01 | g |
| MCT | 37% | 3.7 | g |
| HS15 | 43.9% | 4.39 | g |
| EPC | 3% | 0.3 | g |
| Absolute ethanol | 16% | 1.6 | g |
| In total | | 10 | g |

Process:

EPC, levosimendan, absolute ethanol, MCT and HS15 in the amount specified for the formula above were weighed in order into a 20 ml vial followed by stirring at 2000 rpm in a water bath at for 5 min to give a uniform and transparent oily solution.

Study on Stability of Emulsion:

Liquid concentrate 14 was diluted with 5% glucose injection in a ratio of 1 g:50 ml. The particle size and stability of the resulting emulsion were examined for 24 hours.

| Item | 0 h | 24 h |
|---|---|---|
| Appearance | Emulsion with | Emulsion with obvious |
| | obvious slightly blue | slightly blue opalescence, |
| | opalescence | no drug precipitation |
| Average particle | 135.27 | 131.46 |
| size (nm) | | |
| PDI | 0.17 | 0.19 |

### Liquid Concentrate 15

Formula:

| Component | % by weight * | Amount | |
|---|---|---|---|
| Clevidipine butyrate | 2% | 0.2 | g |
| MCT | 28% | 2.8 | g |
| HS15 | 48% | 4.8 | g |
| EPC | 4% | 0.4 | g |
| Absolute ethanol | 17.9% | 1.79 | g |
| Ascorbyl palmitate | | 0.01 | g |
| In total | | 10 | g |

Process:

EPC, clevidipine butyrate, absolute ethanol. MCT and HS15 in the amount specified for the formula above were weighed in order into a 20 ml vial followed by stirring at 2000 rpm in a water bath at 60° C. for 5 min to give a uniform and transparent oily solution.

Study on Stability of Emulsion:

Liquid concentrate 15 was diluted with 5% glucose injection in a ratio of 1 g:50 ml. The particle size and stability of the resulting emulsion were examined for 24 hours.

| Item | 0 h | 24 h |
|---|---|---|
| Appearance | Emulsion with | Emulsion with obvious |
| | obvious slightly blue | slightly blue opalescence, |
| | opalescence | no drug precipitation |
| Average particle | 39.58 | 41.37 |
| size (nm) | | |
| PDI | 0.11 | 0.12 |

### Liquid Concentrate 16

Formula:

| Component | % by weight * | Amount |
|---|---|---|
| Clopidogrel | 2% | 0.2 g |
| MCT | 32% | 3.2 g |
| HS15 | 47% | 4.7 g |
| EPC | 5% | 0.5 g |
| Absolute ethanol | 14% | 1.4 g |
| In total | | 10 g |

Process:

EPC, clopidogel, absolute ethanol, MCT and HS15 in the amount specified for the formula above were weighed in order into a 20 ml vial followed by stirring at 2000 rpm in a water bath at 60° C. for 5 min to give a uniform and transparent oily solution.

Study on Stability of Emulsion:

Liquid concentrate 16 was diluted with 5% glucose injection in a ratio of 1 g:100 ml. The particle size and stability of the resulting emulsion were examined for 24 hours.

| Item | 0 h | 24 h |
|---|---|---|
| Appearance | Emulsion with | Emulsion with obvious |
| | obvious slightly blue | slightly blue opalescence, |
| | opalescence | no drug precipitation |
| Average particle | 100.25 | 101.28 |
| size (nm) | | |
| PDI | 0.12 | 0.13 |

### Liquid Concentrate 17

Formula:

| Component | % by weight * | Amount |
|---|---|---|
| Remdesivir | 1% | 0.1 g |
| MCT | 37% | 3.7 g |
| HS15 | 43% | 4.3 g |
| EPC | 3% | 0.3 g |
| Absolute ethanol | 16% | 1.6 g |
| In total | | 10 g |

Process:

EPC and remdesivir in the amount specified for the formula above were weighed into a 20 ml vial. Absolute ethanol in the amount specified for the formula above was added followed by stilling at 2000 rpm in a water bath at 60° C. for 1 min. MCT and HS15 in the amount specified for the formula above were added followed by stirring at 2000 rpm in a water bath at 60° C. for 5 min to give a uniform and transparent oily solution.

Study on Stability of Emulsion:

Liquid concentrate 17 was diluted with 5% glucose injection in a ratio of 1 g:100 ml. The particle size and stability of the resulting emulsion were examined for 24 hours.

| Item | 0 h | 24 h |
|---|---|---|
| Appearance | White emulsion | White emulsion, no drug |
| | | precipitation |
| Average particle size (nm) | 183.21 | 181.22 |
| PDI | 0.21 | 0.25 |

### Liquid Concentrate 18

Formula:

| Component | % by weight * | Amount |
|---|---|---|
| Tacrolimus | 1% | 0.1 g |
| MCT | 28% | 2.8 g |
| HS15 | 50% | 5.0 g |
| EPC | 3% | 0.3 g |
| Absolute ethanol | 18% | 1.8 g |
| In total | | 10 g |

Process:

EPC in the amount specified for the formula above was weighed into a 20 ml vial. Absolute ethanol in the amount specified for the formula above was added followed by stirring at 2000 rpm in a water bath at 60° C. for 1 min to dissolve EPC. Tacrolimus in the amount specified for the formula above was added followed by stirring at 2000 rpm in a water bath at 60° C. for 1 mM. HS15 and MCT in the amount specified for the formula above were added followed by stirring at 2000 rpm in a water bath at 60° C. for 5 min to give a uniform and transparent oily solution.

Study on Stability of Emulsion:

Liquid concentrate 18 was diluted with 5% glucose injection in a ratio of 1 g:50 ml. The particle size and stability of the resulting emulsion were examined for 24 hours.

| Item | 0 h | 24 h |
|---|---|---|
| Appearance | Emulsion with | Emulsion with obvious |
| | obvious slightly blue | slightly blue opalescence, |
| | opalescence | no drug precipitation |
| Average particle | 29.11 | 31.28 |
| size (nm) | | |
| PDI | 0.01 | 0.01 |

### Liquid Concentrate 19

Formula:

| Component | % by weight * | Amount |
|---|---|---|
| Paclitaxel | 2% | 0.2 g |
| MCT | 40% | 4.0 g |
| ELP | 33% | 3.3 g |
| EPC | 5% | 0.5 g |
| Absolute ethanol | 20% | 2.0 g |
| In total | | 10 g |

Process:

EPC in the amount specified for the formula above was weighed into a 20 ml vial. Absolute ethanol in the amount specified for the formula above was added followed by stirring at 2000 rpm in a water bath at 60° C. for 1 min to dissolve EPC. Paclitaxel in the amount specified for the formula above was added followed by stirring at 2000 rpm in a water bath at 60° C. for 1 min. ELP and MCT in the amount specified for the formula above were added followed by stirring at 2000 rpm in a water bath at 60° C. for 5 min to give a uniform and transparent oily solution.

Study on Stability of Emulsion:

Liquid concentrate 19 was diluted with 5% glucose injection in a ratio of 1 g:50 ml. The particle size and stability of the resulting emulsion were examined for 24 hours.

| Item | 0 h | 24 h |
|---|---|---|
| Appearance | White emulsion | White emulsion, no drug |
| | | precipitation |
| Average particle size (nm) | 355.69 | 361.42 |
| PDI | 0.24 | 0.21 |

### Liquid Concentrate 20

Formula:

| Component | % by weight * | Amount |
|---|---|---|
| Ibuprofen | 12% | 1.2 g |
| MCT | 27% | 2.7 g |
| HS15 | 48% | 4.8 g |
| EPC | 2% | 0.2 g |
| Absolute ethanol | 11% | 1.1 g |
| In total | | 10 g |

Process:

EPC, ibuprofen, absolute ethanol. MCT and HS15 in the amount specified for the formula above were weighed in order into a 20 ml vial followed by stirring at 2000 rpm in a water bath at 60° C. for 5 min to give a uniform and transparent oily solution.

Study on Stability of Emulsion:

Liquid concentrate 20 was diluted with 5% glucose injection in a ratio of 1 g:50 ml. The particle size and stability of the resulting emulsion were examined for 24 hours.

| Item | 0 h | 24 h |
|---|---|---|
| Appearance | White emulsion | White emulsion, no drug |
| | | precipitation |
| Average particle size (nm) | 38.4 nm | 40.6 |
| PDI | 0.04 | 0.03 |

### Liquid Concentrate 21

Formula:

| Component | % by weight * | Amount |
|---|---|---|
| Carboplatin | 1% | 0.1 g |
| MCT | 32% | 3.2 g |
| Tween 80 | 48% | 4.8 g |
| SPC | 3% | 0.3 g |
| Absolute ethanol | 16% | 1.6 g |
| In total | | 10 g |

Process:

SPC, carboplatin, absolute ethanol. MCT and Tween 80 in the amount specified for the formula above were weighed in order into a 20 ml vial followed by stirring at 2000 rpm in a water bath at 60° C. for 5 min to give a uniform and transparent oily solution.

Study on Stability of Emulsion:

Liquid concentrate 21 was diluted with 5% glucose injection in a ratio of 1 g:50 ml. The particle size and stability of the resulting emulsion were examined for 24 hours.

| Item | 0 h | 24 h |
|---|---|---|
| Appearance | Emulsion with | Emulsion with obvious |
| | obvious slightly blue | slightly blue opalescence, |
| | opalescence | no drug precipitation |
| Average particle | 169.54 | 171.41 |
| size (nm) | | |
| PDI | 0.18 | 0.20 |

### Liquid Concentrate 22

Formula:

| Component | % by weight * | Amount |
|---|---|---|
| Dexamethasone palmitate | 1% | 0.1 g |
| MCT | 37% | 3.7 g |
| HS15 | 43% | 4.3 g |
| EPC | 3% | 0.3 g |
| Absolute ethanol | 16% | 1.6 g |
| In total | | 10 g |

Process:

EPC, dexamethasone palmitate, absolute ethanol, MCT and HS15 in the amount specified for the formula above were weighed in order into a 20 ml vial followed by stirring at 2000 rpm in a water bath at 60° C. for 5 min to give a uniform and transparent oily solution.

Study on Stability of Emulsion:

Liquid concentrate 22 was diluted with 5% glucose injection in a ratio of 1 g:50 ml. The particle size and stability of the resulting emulsion were examined for 24 hours.

| Item | 0 h | 24 h |
|---|---|---|
| Appearance | Emulsion with | Emulsion with obvious |
| | obvious slightly blue | slightly blue opalescence, |
| | opalescence | no drug precipitation |
| Average particle | 129.33 | 131.16 |
| size (nm) | | |
| PDI | 0.15 | 0.16 |

### Liquid Concentrate 23

Formula:

| Component | % by weight * | Amount |
|---|---|---|
| Valdecoxib | 5% | 0.5 g |
| MCT | 28% | 2.8 g |
| HS15 | 48% | 4.8 g |
| EPC | 1% | 0.1 g |
| Absolute ethanol | 18% | 1.8 g |
| In total | | 10 g |

Process:

EPC, valdecoxib, absolute ethanol, MCT and HS15 in the amount specified for the formula above were weighed in order into a 20 ml vial followed by stirring at 2000 rpm in a water bath at for 5 min to give a uniform and transparent oily solution.

Study on Stability of Emulsion:

Liquid concentrate 23 was diluted with 5% glucose injection in a ratio of 1 g:50 ml. The particle size and stability of the resulting emulsion were examined for 24 hours.

| Item | 0 h | 24 h |
|---|---|---|
| Appearance | Emulsion with | Emulsion with obvious |
| | obvious slightly blue | slightly blue opalescence, |
| | opalescence | no drug precipitation |
| Average particle | 28.25 | 30.11 |
| size (nm) | | |
| PDI | 0.02 | 0.02 |

### Liquid Concentrate 24

Formula:

| Component | % by weight * | Amount |
|---|---|---|
| Voriconazole | 6% | 0.6 g |
| MCT | 30% | 3.0 g |
| HS15 | 46% | 4.6 g |
| EPC | 4% | 0.4 g |
| Absolute ethanol | 14% | 1.4 g |
| In total | | 10 g |

Process:

EPC, voriconazole, absolute ethanol. MCT and HS15 in the amount specified for the formula above were weighed in order into a 20 ml vial followed by stirring at 2000 rpm in a water bath at 60° C. for 5 min to give a uniform and transparent oily solution.

Study on Stability of Emulsion:

Liquid concentrate 24 was diluted with 5% glucose injection in a ratio of 1 g:50 ml. The particle size and stability of the resulting emulsion were examined for 24 hours.

| Item | 0 h | 24 h |
|---|---|---|
| Appearance | Emulsion with | Emulsion with obvious |
| | obvious slightly blue | slightly blue opalescence, |
| | opalescence | no drug precipitation |
| Average particle | 88.28 | 90.55 |
| size (nm) | | |
| PDI | 0.12 | 0.14 |

### Liquid Concentrate 25

Formula:

| Component | % by weight * | Amount |
|---|---|---|
| Azithromycin | 12% | 1.2 g |
| MCT | 24% | 2.4 g |
| HS15 | 48% | 4.8 g |
| EPC | 2% | 0.2 g |
| Absolute ethanol | 14% | 1.4 g |
| In total | | 10 g |

Process:

EPC, azithromycin, absolute ethanol, MCT and HS15 in the amount specified for the formula above were weighed in order into a 20 ml vial followed by stirring at 2000 rpm in a water bath at for 5 min to give a uniform and transparent oily solution.

Study on Stability of Emulsion:

Liquid concentrate 25 was diluted with 5% glucose injection in a ratio of 1 g:50 ml. The particle size and stability of the resulting emulsion were examined for 24 hours.

| Item | 0 h | 24 h |
|---|---|---|
| Appearance | White emulsion | White emulsion, no drug |
| | | precipitation |
| Average particle size (nm) | 125.56 | 130.13 |
| PDI | 0.14 | 0.18 |

### Liquid Concentrate 26

Formula:

| Component | % by weight * | Amount |
|---|---|---|
| Propofol | 6% | 0.6 g |
| MCT | 35% | 3.5 g |
| HS15 | 48% | 4.8 g |
| EPC | 2% | 0.2 g |
| Absolute ethanol | 9% | 0.9 g |
| In total | | 10 g |

Process:

The individual components in the amount specified for the formula above were weighed in order into a 20 ml vial followed by magnetically stirring until all components were dissolved to give a uniform and transparent oily solution.

Study on Stability of Emulsion:

Liquid concentrate 26 was diluted with 5% glucose injection in a ratio of 1 g:6 ml. The particle size and stability of the resulting emulsion were examined for 24 hours.

| Item | 0 h | 24 h |
|---|---|---|
| Appearance | White emulsion | White emulsion, no drug |
| | | precipitation |
| Average particle size (nm) | 212.16 | 220.38 |
| PDI | 0.11 | 0.15 |

### Liquid Concentrate 27

Formula:

| Component | % by weight * | Amount | |
|---|---|---|---|
| Paclitaxel | 1.5% | 0.15 | g |
| MCT | 29.4% | 2.93 | g |
| HS15 | 49.1% | 4.9 | g |
| EPC | 3% | 0.3 | g |
| Absolute ethanol | 17% | 1.7 | g |
| Citric acid | | 0.02 | g |
| In total | | 10 | g |

Process:

EPC in the amount specified for the formula above was weighed into a 20 ml vial. Absolute ethanol in the amount specified for the formula above was added followed by stirring at 2000 rpm in a water bath at 60° C. for 1 min to dissolve EPC. Paclitaxel in the amount specified for the formula above was added followed by stirring at 2000 rpm in a water bath at 60° C. for linin. HS15 and MCT in the amount specified for the formula above were added followed by stirring at 2000 rpm in a water bath at 60° C. for 5 min to give a uniform and transparent oily solution.

Study on Stability of Emulsion:

Liquid concentrate 27 was diluted with 5% glucose injection in a ratio of 1 g:100 ml. The particle size and stability of the resulting emulsion were examined for 24 hours.

| Item | 0 h | 24 h |
|---|---|---|
| Appearance | White emulsion | White emulsion, no drug |
| | | precipitation |
| Average particle size (nm) | 93.20 | 93.85 |
| PDI | 0.26 | 0.27 |

### Example 5: Study on Stability of the Liquid Concentrates According to the Invention

The stability of liquid concentrate 1 in Example 4 was investigated wider accelerated conditions according to the guidelines for stability tests of active pharmaceutical ingredient and pharmaceutical preparation in Section 9001 of the General Notices, Fourth Part. Chinese Pharmacopoeia 2015.

1. Test product: liquid concentrate 1 prepared in Example 4, batch No. 2020041501

### 2. Conditions for the Stability Tests:

| Item | Condition | Period | Investigated items |
|---|---|---|---|
| Stability test | 40° C. ± 2° C.; | 0, 1, 2, 3, | Description, content, |
| under accelerated | RH60% ± 5% | 6 months | and relevant substances |
| conditions | | | |

### 3. Analysis Method

### (1) Description

Method: Visual Inspection

### (2) Content

Determination Method: High Performance Liquid Chromatography

Chromatographic conditions: chromatographic column: phenyl bonded silica gel as the filler. 250 nm×4.6 mm, 5 um; mobile phase: 2.7 g/L potassium dihydrogen phosphate solution (adjusted with phosphoric acid to pH 3.0)-methanol-acetonitrile (60:30:10); detection wavelength: 215 nm; column temperature: 60° C.; flow rate: 1.5 ml/min; sample injection amount: 20 ul; run time: 30 min.

The specific operations: 12 mg of each of impurity A (4-[5-(3-methylphenyl)-3-(trifluoromethyl)-1H-pyrazol-1-yl]benzenesulfonamide) and impurity B (4-[3-(4-methylphenyl)-5-(trifluoromethyl)-1H-pyrazol-1-yl]benzenesulfonamide) of celecoxib was accurately weighed into the same 100 ml volumetric flask, dissolved and diluted to the scale mark of the volumetric flask with methanol, and shaken up for use as the system control solution. 25 mg of celecoxib standard substance was accurately weighed into a 50 ml volumetric flask, added with 1 ml of the system control solution, dissolved and diluted to the scale mark of the volumetric flask with methanol, and shaken up for use as the system applicability solution. 25 mg of celecoxib standard substance was accurately weighed, dissolved and diluted to the scale mark with methanol, and shaken up for use as reference substance solution. 1.0 g of the test product (liquid concentrate 1 of Example 4) was accurately weighed into a 100 ml volumetric flask, dissolved and diluted to the scale mark of the volumetric flask with methanol, and shaken up for use as the test solution. 20 μl of each of the reference substance solution and the test solution filtered by 0.45 inn nylon membrane was injected to give chromatogram. The contents of celecoxib and impurities were calculated based on the peak area according to an external standard method.

(3) Description. Particle Size, pH Value and Stability of Emulsion

1 g of the liquid concentrate 1 was added into 100 ml of 5% glucose injection. The mixture was manually shaken up to give emulsion. The average particle size and polydispersity index (PDI) of the resulting emulsion were measured using a laser particle sizer (Nicomp Z3000, PSS). The pH value of the emulsion was determined using a pH meter. The emulsion was left to stand at room temperature for 24 h to observe whether there is drug precipitation.

### 4. Experimental Results

TABLE 10
Stability of the liquid concentrate 1 of Example 4

| Investigated item | Requirement | 0 months | 1 month | 2 months | 3 months | 6 months |
|---|---|---|---|---|---|---|
| Appearance | Colorless transparent | Colorless | Colorless | Colorless | Colorless | Colorless |
| | oily solution without | transparent oily | transparent oily | transparent oily | transparent oily | transparent oily |
| | crystallization or | solution without | solution without | solution without | solution without | solution without |
| | precipitation | crystallization of | crystallization or | crystallization of | crystallization or | crystallization of |
| | | precipitation | precipitation | precipitation | precipitation | precipitation |
| Impurities | Not more than 0.7% | 0.48% | 0.51% | 0.56% | 0.53% | 0.57% |
| Content of | 95.0% to 105.0% of | 101.3% | 100.6% | 100.2% | 98.3% | 98.0% |
| celecoxib | the labeled amount | | | | | |

The experimental results of Table 10 showed that liquid concentrate 1 had good physical and chemical stability after standing at 40° C.±2° C., RH60%±5% for 6 months.

TABLE 11
Stability of emulsion formed by the liquid concentrate 1 of Example 4

| Item | Requirement | 0 months | 1 month | 2 months | 3 months | 6 months |
|---|---|---|---|---|---|---|
| Average particle size (nm) | 20-4000 | 30.70 | 32.12 | 30.93 | 33.42 | 30.86 |
| Polydispersity inder (PDI) | 0~0.7 | 0.02 | 0.02 | 0.10 | 0.06 | 0.09 |
| pH | 4.0~9.0 | 4.96 | 4.92 | 5.01 | 5.12 | 4.98 |
| Stability of emulsion | No drug | No drug | No drug | No drug | No drug | No drug |
| formed by the liquid | precipitation in 4 | precipitation in 24 | precipitation in 24 | precipitation in 24 | precipitation in 24 | precipitation in 24 |
| concentrate | hours | hours | hours | bours | hours | bours |

The experimental results of Table 11 showed that during the stability test of 6 months, the liquid concentrate 1 formed emulsion upon dilution with 5% glucose injection, and the average particle size, PDI, and pH value of the resulting emulsion kept stable in 24 h. No layering or drug precipitation occurred. The resulting emulsion had good stability.

### Example 6: Preparation of Liquid Concentrate Comprising Propofol and the Properties Thereof

In this example, the liquid concentrate 26 of Example 4 of the present disclosure was compared with the propofol concentrate B6 of the Chinese patent application CN10834851A and a commercially available medium/long chain fat emulsion of propofol (batch No. 16MC0288. Fresenius Kabi (Beijing)).

The compositions of the liquid concentrate 26 of Example 4 of the present disclosure and the propofol concentrate B6 of Chinese patent application CN10834851A were as follows:

TABLE 12
Formulas of the propofol concentrate B6 of CN10834851A
and the liquid concentrate 26 of the present disclosure

| | Propofol concentrate B6 of | | Liquid concentrate 26 of the |
|---|---|---|---|
| Component | CN10834851A | | present disclosure |
| propofol | 0.6 | g | 0.6 g |
| HS15 | 7.0246 | g | 4.8 g |
| EPC | 0.188 | g | 0.2 g |
| MCT | 0.752 | g | 3.5 g |
| Ethanol | 0.4954 | g | 0.9 g |
| Propylene | 0.188 | g | — |
| glycol | | | |
| PEG400 | 0.752 | g | — |
| In total | 10 | g | 10 g |

Process: Individual components in the amount specified for each formula were added into a 20 ml vial and was magnetically stirred until all components completely dissolved.

The two concentrates of propofol obtained were both uniform and transparent oily solutions.

The obtained oily solutions were diluted with 5% glucose injection in a ratio of 1 g:6 ml to the propofol concentration of 10 mg/ml.

The average particle size and PDI were measured with a laser particle sizer (NicompZ3000. PSS. USA). The free drug was separated by centrifugation using 3KD ultrafiltration tube (Millipore). The concentration of the free drug was determined with HPLC (instrument model: LC-20AT Shimadzu; chromatographic column: Agilent Zorbaxextend C18 (250 mm×4.6 mm, 5 um); mobile phase: sodium dihydrogen phosphate solution (pH3.0)-acetonitrile); detection wavelength: 275 nm; column temperature: 40° C.; flow rate: 1.0 ml/min; sample injection amount: 10 ul.

In addition, the hemolysis rate was determined by in vitro hemolysis method (2% rabbit red blood cell suspension). The specific protocol was as follows:

Preparation of Negative Control Tube

A tube was taken and added with 2.0 ml of 2% erythrocyte suspension and 2.0 ml of 5% glucose injection.

Preparation of Positive Control Tube

A tube was taken, added with 2.5 ml of 2% erythrocyte suspension and centrifugated (1500 r/min) for 5 min. The 1.5 ml of colorless supernatant was discarded. 4 ml of distilled water was added to the residue to break erythrocytes to give the positive control tube with complete hemolysis.

Preparation of the Test Product Tube

A tube was taken, added with 0.3 ml of the liquid concentrate of propofol, 2.2 ml of 5% glucose injection and 2.5 ml of 2% erythrocyte suspension, and shaken up for use as the test product tube. The test product was prepared in duplicate.

Determination of Samples

Individual tubes were incubated in a water bath at 37±0.5° C. for 3 h, and then centrifugated (1500 r/min) for 5 min. 0.2 ml of supernatant was accurately taken and added accurately with a demulsifier (0.2 ml of concentrated hydrochloric acid was pipetted, added with 79.8 ml of absolute ethanol, and mixed uniformly) to give 3.2 ml of mixture (demulsification ratio 16:1). The mixture was shaken up, and taken in an appropriate amount for measurement of the absorbance of heme at 400 nm (instrument: ultraviolet spectrophotometer, model: T6 new century, manufacturer: Beijing Puxi General Instrument Co., Ltd; wavelength: 400 nm). The hemolysis rate was calculated according to the equation below:

Hemolysis rate (%)=(absorbance of the test product tube−absorbance of the negative control tube)/(absorbance of the positive control tube−absorbance of the negative control tube)×100%

The experimental results were shown in Table 13.

TABLE 13
Comparison of properties of the propofol concentrate
B6 of CN10834851A, the liquid concentrate 26
of the present disclosure, and the commercially
available fat emulsion of propofol

| | Average Particle | Free | Hemolysis |
|---|---|---|---|
| Formulation | Size (MD)/PDI | propofol (%) | rate (%) |
| Propofol concentrate B6 of | 16.88 nm/0.06 | 0.6 | 31.62 |
| CN10834851A | | | |
| Liquid concentrate 26 of | 212.16 nm/0.11 | 0.01 | 2.25 |
| the present disclosure | | | |
| Commercially available | 217.29 nm/0.22 | 0.02 | 2.30 |
| medium/long chain fat | | | |
| emulsion of propofol | | | |

As can be seen from the experimental results in Table 13, the emulsion obtained upon dilution of the propofol concentrate B6 of CN10834851A was a uniform and transparent solution with the average particle size of 16.88 nm, which was similar to a true solution. The emulsion was stable after standing at room temperature for 24 h. The amount of free propofol in the emulsion was as high as 0.6%, resulting in an in vitro hemolysis rate of up to 31.62%.

The liquid concentrate 28 of the present disclosure formed a white uniform emulsion upon dilution with an average particle size of 212.16 nm. The emulsion remained stable after standing at room temperature for 24 h. The amount of free propofol in the emulsion was only 0.01%, and the in vitro hemolysis rate was only 2.25%.

It is known in the art that the amount of free propofol in a formulation is the major cause of injection pain and also of in vitro hemolysis, and the hemolysis rate caused by propofol is in the range of 3.1% to 58% with a good linear relationship (r=0.9650) to the concentration of free propofol (see, for example, CAI Weihui and JIN Fang, “Relationship between Free Propofol Concentrations of Different Delivery Systems for Injection and in vitro Hemolytic Activities”. Chinse Journal of Pharmaceuticals, 2008, 39(1) 22-26). Thus, compared with the propofol concentrate B6 of CN10834851A, the liquid concentrate 26 of the present disclosure reduced the number of excipients while maintaining good stability, and thus had reduced adverse effects (i.e., less injection pain and lower hemolysis rate).

In addition, the emulsion formed by diluting the liquid concentrate 26 of the present disclosure was similar to the commercially available medium/long-chain fat emulsion of propofol for injection in terms of appearance, average particle size, PDI (PDI) and hemolysis rate, fully met the requirements of intravenous injection administration, and avoided the complicated preparation process and strict conditions for the storage and transportation of the fat emulsion injection.

### Example 7: Pharmacodynamic Study of Celecoxib Liquid Concentrate in Rat Model of Plantar Incisional Pain

### 1. Materials

### 1.1 Test Product

Name: celecoxib injection

Preparation method: 2.1 g of the liquid concentrate 2 of Example 4 (batch No. 2020060401) was extracted with 2.5 ml syringe, and injected into 100 ml of 5% glucose injection, manually shaken up to give emulsion. The properties of the resulting emulsion were as follows:

| Concentration of celecoxib | MD | PDI |
|---|---|---|
| 1.05 mg/mL | 33.42 nm | 0.03 |

### 1.2 Positive Control

(1) Positive Control 1

Name: parecoxib sodium injection

Batch No.: CJ2725

Strength: 40 mg/piece

Description: white lyophilized powder

Expiry: March 2022

Manufacturer: Pfizer

The positive control 1 was prepared by diluting with 5% glucose injection to a concentration of 0.41 mg/ml of paroxycoxib sodium.

(2) Positive Control 2

Name: flurbiprofen axetil injection (Kaifen®)

Batch No.: 3E309E

Strength: 5 ml:50 mg

Dosage form: injection

Expiry: Mar. 20, 2021

Manufacturer: Beijing Tide Pharmaceutical

The positive control 2 was prepared by diluting with 5% glucose injection to a concentration of 1.05 mg/ml of flurbiprofen axetil.

### 1.3 Negative Control

Name: 5% glucose injection

Batch No.: 1909061B

Manufacturer: Anhui Double-Crane Pharmaceutical Co., Ltd.

### 1.4 Blank Formulation

Name: blank formulation (The only difference of blank formulation from the test product is that the poorly soluble drug is replaced with the same weight of absolute ethanol, and the other components and the preparation method are the same as the test product)

Batch No.: 2020060901

Strength: 0 mg/bottle

Concentration: 0 mg/ml

Description: Clear and transparent solution

Manufacturer: Beijing Delivery Pharmaceutical Technology Co., Ltd.

### 1.5 Animals

Strain: SD rat

Grade: SPF grade animal

Gender and number: male. 60 rats

Age: about 6-8 weeks

Weight: 220-260 g

Source: Beijing FMK Bioscience Co., Ltd.

### 2. Experimental Design

The dosages of the commercially available paroxicib sodium injection and flurbiprofen axetil injection (Kaifen®) in humans were converted to the dosages in inflammatory model of animals (rats), which were 8.4 mg/kg and 21 mg/kg, respectively.

The test product, the two positive controls, the negative control and the blank formulation were given once by intravenous injection according to the concentration and dosage indicated in Table 14.

### 2.1 The Preparation of the Rat Model of Plantar Incision

Plantar incision model: the male SD rats described in 1.4 above were kept under laboratory conditions for 1 week. After rats were anesthetized by inhalation of isoflurane, an incision with a length of about 1 cm was made from the proximal 0.5 cm of the planta pedis to the toe by incising the skin and fascia, then picking up the plantar muscle using ophthalmologic forceps and longitudinally cutting (keeping the muscle and attachments thereof complete), and stopping bleeding by pressing. Saline and the individual articles were injected subcutaneously near the plantar incision of the animals. Pain threshold test was then performed by the hotplate method. This rat model was used to simulate postoperative pain in clinic.

### 2.2 Dosage and Grouping

The successfully molded SD rats were randomly divided into 5 groups with 10 rats each group. The 5 groups were used as parecoxib sodium group (group A), celecoxib injection group (group B), flurbiprofen axetil injection (Kaifen®) group (group C), negative control group (group D) and blank formulation group (group E), and were given with the corresponding articles once by tail vein infusion, respectively. The detailed dosing regimen was shown in Table 14.

TABLE 14
Dosing regimen of pharmacodynamic study in SD rats

| | Administered | Administration | Dosage | Concentration | Volume | |
|---|---|---|---|---|---|---|
| Group | formulation | mode | (mg/kg) | (mg/ml) | (ml/100 g) | Animal |
| A | Parecoxib sodium | IV | 8.4 | 0.41 | 2.0 | 10 |
| B | Celecoxib injection | IV | 21 | 1.05 | 2.0 | 10 |
| C | flurbiprofen axetil | IV | 21 | 1.05 | 2.0 | 10 |
| | injection | | | | | |
| D | Negative control | IV | 0 | 0 | 2.0 | 10 |
| E | Blank formulation | IV | 0 | 0 | 2.0 | 10 |

Notes:
(1) group D was the negative control group, and 5% glucose injection was given to the rats in this group;
(2) group E was the blank formulation group, and the blank formulation given to the rats in this group was the emulsion obtained upon dilution of blank celecoxib injection (i.e., the same emulsion as group B without celecoxib, wherein celecoxib was replaced by the same weight of absolute ethanol);
(3) the administration mode of all groups was intravenous infusion using an injection pump, wherein the infusion lasted for 25-30 min.

### 2.3 Detection Frequency and Method

Evaluation of sensory blockade: von Frey hair (Stoelting, Wood Dale, USA) was used to evaluate the sensory blockade, and the procedures were the same as those for determining the pain threshold for screening and grouping of animals. The administration side was detected using von Frey hair at 30 min, 1 h, 2 h, 4 h, and 8 h after the administration.

In order to evaluate the rat pain condition, mechanical hyperalgesia was assessed using Von Frey fine filaments in a quiet environment at ambient temperature (22±1° C.).

During the measurement, the rats were placed in a tailored organic glass box with a grid bottom (26 cm×20 cm×14 cm) or in a steel wire grid so as to limit the free movement of the rats within a small range. After the rats were adapted for 20 min, a series of von Frey filaments (0.4, 0.6, 1.4, 2.0, 4.0, 6.0, 8.0 and 15.0 g) were used for stimulating the skin in the middle of the rat voix pedis at the side of administration starting from 2.0 g of force according to the up and down method introduced by Dixon. The filaments were pressed on the voix pedis to be C-shaped so that the arc was like the ⅜ suture needle for timing until the animal lifted the foot or walked away. The rat paw withdrawal response was observed.

No response is designated as negative response “O”, and the response of paw withdrawal or licking is designated as positive response “X”. If there is not response to the stimulation of the first filament, the stimulation of the next greater filament will be given, or if there is a response to the stimulation of the first filament, the stimulation of the next less filament will be given. This process is repeated until the first positive and negative (or negative and positive) response occur. Then, continuous determination is carried out for 4 times. Starting from the “O” response just before the occurrence of the “X” response, the OX values obtained by 6 consecutive stimulations including the starting point itself are selected as the key series for the estimation of the 50% withdrawal threshold. Said 6 stimulations are not mandatory, and the number of stimulations may be a minimum of 4 and a maximum of 9. The interval of stimulations is 30 seconds so as to eliminate the influence of the previous stimulation. If 50% withdrawal threshold does not occur in the rats until the maximum von Frey filament of 15.00 g is tested, the 50% withdrawal threshold will be recorded as 15.00 g. After a series of combinations of “O” or “X” are obtained, the mechanical pain threshold is calculated based on the series and the force (f) of the last filament using the following formula.

50% withdrawal threshold (g)=(10[Xf+kδ])/10 000
Wherein.
Xf=log(f*10000);
δ is the mean difference of logarithm of the filament force, and is equal to about 0.224 here;
k is a value obtained by looking up a table according to the measured X and O series.

In order to avoid the animal tolerance or pain sensitivity caused by frequent or long-term stimulation, each filament has a maximum stimulation time of not more than 8 seconds, and mechanical hyperalgesia is measured at interval of more than 4 d in the experiments.

### 3. Experimental Results

TABLE 15
50% withdrawal threshold of individual groups
50% withdrawal threshold (g)

| | Parecoxib | Flurbiprofen | | Negative | Blank |
|---|---|---|---|---|---|
| Time (h) | sodium | axetil | Celecoxib | control | formulation |
| 0.5 | 6.0 ± 3.21 | 8.43 ± 3.23 | 13.39 ± 3.41 | 3.3 ± 1.80 | 3.4 ± 1.00 |
| 1 | 6.9 ± 3.08 | 7.82 ± 4.38 | 13.89 ± 2.41 | 3.8 ± 1.50 | 4.4 ± 2.63 |
| 2 | 8.4 ± 2.94 | 8.42 ± 2.78 | 14.29 ± 2.25 | 4.4 ± 2.9 | 4.8 ± 1.74 |
| 4 | 8.1 ± 3.88 | 9.13 ± 3.79 | 14.27 ± 2.00 | 4.6 ± 4.4 | 4.3 ± 1.79 |
| 8 | 8.9 ± 4.30 | 9.75 ± 4.52 | 14.66 ± 1.08 | 3.9 ± 2.1 | 6.8 ± 2.74 |

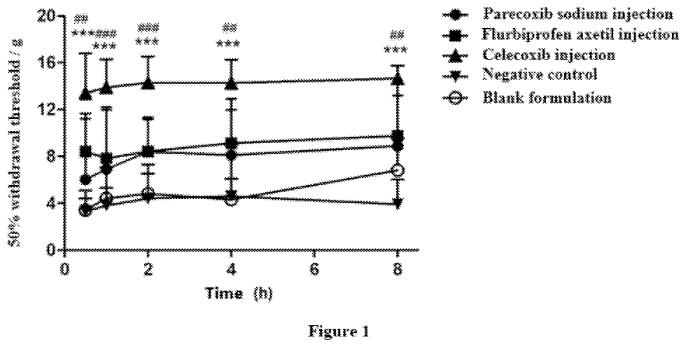

The data In table 15 were plotted to give FIG. 1, in which the first curve (▴) represented the experimental results of the celecoxib emulsion, the second curve (▪) represented the experimental results of the flurbiprofen axetil injection, the third curve (●) represented the experimental results of the parecoxib sodium injection, the fourth curve (∘) represented the experimental results of the blank formulation, and the fifth curve (▾) represented the experimental results of the negative control (5% glucose injection). As can be clearly seen from FIG. 1, in the rat plantar incisional pain model, compared with the commercially available parecoxib sodium injection and flurbiprofen axetil injection, the celecoxib emulsion according to the invention had stronger analgesic effect and significant difference (one-way analysis of variance (ANOVA) test using GraphPad Prism software, *** represented that the celecoxib emulsion injection according to the invention had significant difference compared with the parecoxib sodium injection p<0.01. ## represented that the celecoxib emulsion according to the invention had significant difference compared with the flurbiprofen axetil injection p<0.05, and ### represented that the celecoxib emulsion according to the invention had significant difference compared with the flurbiprofen axetil injection p<0.01). In conclusion, the celecoxib emulsion injection has better therapeutic effect.

### Example 8: Evaluation on the Efficacy of the Liquid Concentrate Comprising Ibuprofen on Carrageenan-Induced Paw Swelling in Rats

The administration of the commercially available ibuprofen injection was determined as follows: for pain treatment of adults, the administration was performed in the dosage of 400-800 mg once according to needs, and the intravenous infusion lasted for more than 30 min. The concentration of ibuprofen for infusion after dilution was 4 mg/ml or less. The maximum dosage administered was 3.2 g per day.

The dosage of the commercially available ibuprofen injection in humans was converted to the dosage for the inflammation model animals (rats). Both the ibuprofen injection prepared according to the present disclosure and the commercially available ibuprofen injection were prepared to the corresponding concentrations for administration.

### 1. Materials

### 1.1 Test Product

Name: ibuprofen injection

Preparation: 0.75 g of the liquid concentrate 20 of Example 4 (batch No. 2020030501) was diluted with 25 ml of 5% glucose injection to a concentration of ibuprofen of 3.6 mg/ml to give an emulsion. The properties of the resulting emulsion were as follows:

| Ibuprofen concentration | MD | PDI |
|---|---|---|
| 1.05 mg/mL | 38.4 nm | 0.04 |

### 1.2 Positive Control

Name: commercially available ibuprofen injection (Caldolor)

Batch No.: 005A19A

Strength: 800 mg/piece

Concentration: 100 mg/ml

Description: clear and transparent oily solution

Storage condition and stability: storage at 25° C. in dark. Two-year shelf life at room temperature.

Expiry: 09/2025

Manufacturer: Cumberland Pharmaceuticals

Preparation of the positive control: the commercially available ibuprofen injection, Caldolor, was diluted with 5% glucose injection to a concentration of ibuprofen of 3.6 mg/ml.

### 1.3 Negative Control

Name: 5% glucose injection

Preparation of the negative control: it can be used directly without preparation.

### 1.4 Animals

Strain: SD rat

Grade: SPF grade animal

Gender and number: male. 30 rats

Age: about 6-8 weeks

Weight: 160-180 g

Source: Beijing HFK (Bioscience Co., Ltd.

### 2. Experimental Design

### 2.1 Preparation of the SD Rat Food Swelling Model and Administration

The toe volume was measured after the animals were grouped. The corresponding articles were given. After 30 min, 100 microliter of carrageenan solution with the concentration of 10 mg/ml was injected in a multi-point way, and the toe volume was continuously measured for 8 hours after 20 min of the injection of carrageenan.

### 2.2 Dosage and Grouping

Dosage: the dosage for humans (400 mg/time/70 kg) was converted to the dosage of 36 mg/kg for rats.

Grouping: the SD rats were randomly divided into 3 groups with 10 animals each group. The 3 groups were used as the commercially available preparation group (group A, the positive control, the administration concentration of 3.6 mg/ml), the ibuprofen injection group (group B, the liquid concentrate 20 of Example 4 diluted with 5% glucose injection to an administration concentration of 3.6 mg/ml), and the negative control group (group C, 5% glucose injection), and were administered once by tail vein injection. The detailed dosing regimen was shown in Table 16.

TABLE 16
Dosing regimen

| | | | | Adminis- | |
|---|---|---|---|---|---|
| | Dosage | Concentration | Volume | tration | Number of |
| Group | (mg/kg) | (mg/ml) | (ml/100 g) | frequency | animals |
| A | 36 | 3.6 | 1 | once | 10 |
| B | 36 | 3.6 | 1 | | 10 |
| C | 0 | 0 | 1 | | 10 |

### 2.3 Dosing Regimen

Injection by tail vein without recovery period.

### 2.4 Detection Frequency and Method

### (1) Collection of Pharmacodynamic and Pharmacokinetic Parameters

The toe volume was measured at 20 min, 40 min, 1 h, 2 h, 3 h, 4 h, 5 h, 6 h, 8 h after carrageenan injection.

After the experiment, the foot tissue was taken and homogenized for analysis and detection.

### 3. Experimental Results

### 3.1 General Observations

After the rats were injected with carrageenan, they had swollen paw and reduced activity.

### 3.2 Change of Swelling Volume Differences

The changes of volume difference of paw swelling of individual groups were shown in Table 17.

TABLE 17
Volume differences of paw swelling at different time
points for each group (X̅ ± SD)

Swelling volume difference (mL)

| | | | |
|---|---|---|---|
| Time | Group A | Group B | Group C |

| | | | | |
|---|---|---|---|---|
| 0 | min | 0.00 ± 0.00 | 0.00 ± 0.00 | 0.00 ± 0.00 |
| 20 | min | 0.19 ± 0.05 | 0.10 ± 0.06* | 0.23 ± 0.07 |
| 40 | min | 0.16 ± 0.04 | 0.07 ± 0.03** | 0.21 ± 0.07 |
| 1 | h | 0.12 ± 0.02* | 0.06 ± 0.04** | 0.20 ± 0.07 |
| 2 | h | 0.14 ± 0.04** | 0.11 ± 0.05** | 0.28 ± 0.08 |
| 3 | h | 0.07 ± 0.01** | 0.11 ± 0.03** | 0.34 ± 0.12 |
| 4 | h | 0.10 ± 0.04** | 0.16 ± 0.02** | 0.37 ± 0.10 |
| 5 | h | 0.14 ± 0.03** | 0.17 ± 0.02** | 0.33 ± 0.08 |
| 6 | h | 0.13 ± 0.04** | 0.16 ± 0.05** | 0.38 ± 0.06 |
| 8 | h | 0.08 ± 0.08** | 0.13 ± 0.04** | 0.29 ± 0.1 |

*P < 0.05 compared with group C (the negative control group); **P < 0.01 compared with group C (the negative control group), and group A was not significantly different from group B.

### 3.3 Change of Swelling Percentage

TABLE 18
Paw swelling at different time points for each group (X̅ ± SD)

Swelling %

| | | | |
|---|---|---|---|
| Time point | Group A | Group B | Group C |

| | | | | |
|---|---|---|---|---|
| 20 | min | 8.92 ± 2.66 | 5.29 ± 2.95* | 11.47 ± 4.16 |
| 40 | min | 7.83 ± 2.05 | 3.60 ± 1.57** | 10.56 ± 4.06 |
| 1 | h | 5.98 ± 1.48 | 3.22 ± 2.13* | 10.36 ± 4.04* |
| 2 | h | 6.88 ± 2.52 | 5.43 ± 2.74 | 14.33 ± 4.11** |
| 3 | h | 3.60 ± 0.67 | 5.70 ± 1.43* | 17.06 ± 6.16** |
| 4 | h | 5.00 ± 1.87 | 7.97 ± 1.38* | 18.74 ± 5.10** |
| 5 | h | 6.81 ± 2.03 | 8.46 ± 1.12 | 16.78 ± 4.36** |
| 6 | h | 6.36 ± 2.17 | 8.15 ± 2.77 | 19.17 ± 3.44** |
| 8 | h | 4.23 ± 3.98 | 6.43 ± 2.25 | 14.88 ± 5.28** |

*P < 0.05 compared with group A (the commercially available ibuprofen injection group)
**P < 0.01 compared with group A (the commercially available ibuprofen injection group)

### 4. Conclusion

The effect of inhibiting rat paw swelling caused by carrageenan was observed in the positive control group and the ibuprofen injection group. The ibuprofen injection group had significant effect of inhibiting the paw swelling at 40 min to 8 hours after administration compared with the negative control group and had comparable anti-inflammatory effect with the commercially available ibuprofen injection group.

All patent and non-patent documents cited herein are incorporated by reference in their entirety as if each is individually set forth.

Although specific embodiments and examples are provided herein to illustrate the invention, they are not intended to limit the scope of the invention. Based on the disclosure, it will be obvious to those skilled in the art to obtain other modifications and equivalents without departing from the spirit of the invention, and those modifications and equivalents are also within the scope of the invention.

1. A liquid concentrate, characterized in that, the liquid concentrate comprises a poorly soluble drug and a self-emulsifying carrier consisting of:
(1) A composite emulsifier consisting of phospholipid and a non-phospholipid emulsifier;
(2) An oil, which is medium chain triglyceride; and
(3) A co-emulsifier, which is absolute ethanol,
wherein the phospholipid is selected from the group consisting of soybean phospholipid, egg yolk lecithin, and a mixture thereof.

2. The liquid concentrate according to claim 1, wherein the non-phospholipid emulsifier is selected from the group consisting of polyoxyethylene castor oil (e.g., polyoxyethylene 35 castor oil, pure polyoxyethylene 35 castor oil), polyoxyethylene hydrogenated castor oil (e.g., polyoxyethylene 40 hydrogenated castor oil, polyoxyethylene 60 hydrogenated castor oil), polyethylene glycol 15-hydroxystearate, D-alpha-tocopheryl polyethylene glycol 1000 succinate (TPGS), polysorbates (e.g., polysorbate 20, 21, 40, 60, 61, 65, 80, 81, 85, 120, particularly polysorbate 80), and a mixture thereof.

3. The liquid concentrate according to claim 2, wherein the non-phospholipid emulsifier is selected from the group consisting of polyoxyethylene 40 hydrogenated castor oil, polyoxyethylene 35 castor oil, pure polyoxyethylene 35 castor oil, polyethylene glycol 15-hydroxystearate, polysorbate 80, and a mixture thereof.

4. The liquid concentrate according to claim 1, wherein when the weight of the poorly soluble drug, the composite emulsifier, the oil and the co-emulsifier is regarded as 100%, the poorly soluble drug accounts for 0.01% to 20% by weight, preferably 0.1% to 15% by weight, more preferably 0.1% to 12% by weight; the phospholipid accounts for 0.5% to 10% by weight, preferably 1% to 5% by weight; the non-phospholipid emulsifier accounts for 30% to 70% by weight, preferably 30% to 60% by weight, more preferably 30% to 50% by weight; and the medium chain triglyceride accounts for 20% to 50% by weight, preferably 20% to 40% by weight, more preferably 23% to 40% by weight; and the absolute ethanol accounts for the balance.

5. The liquid concentrate according to claim 1, wherein the poorly soluble drug is selected from the group consisting of celecoxib, valdecoxib, etocoxib, ibuprofen, dexibuprofen, propofol, flurbiprofen axetil, alprostadil, clevidipine butyrate, dexamethasone palmitate, felodipine, nimodipine, nifedipine, nitrendipine, cyclosporine, tacrolimus, levosimendan, adefovir dipivoxil, erythromycin, roxithromycin, posaconazole, itraconazole, voriconazole, miconazole, ketoconazole, progesterone, coenzyme Q10, clopidogrel, paclitaxel, docetaxel, cabazitaxel, etoposide, teniposide, hydroxycamptothecin, irinotecan, ubenimex, cisplatin, carboplatin, capecitabine, oxaliplatin, gefitinib, doxorubicin, vinblastine, vincristine, vinpocetine, vindesine, piroxicam, spironolactone, valproic acid, tamoxifen, azithromycin, vitamin A, vitamin D, vitamin E, vitamin K, fenofibrate, indomethacin, and remdesivir; preferably, the poorly soluble drug is selected from the group consisting of celecoxib, ibuprofen, dexibuprofen, propofol, flurbiprofen axetil, alprostadil, clevidipine butyrate, dexamethasone palmitate, felodipine, nimodipine, nifedipine, nitrendipine, cyclosporine, tacrolimus, levosimendan, adefovir dipivoxil, erythromycin, roxithromycin, posaconazole, itraconazole, voriconazole, progesterone, coenzyme Q10, clopidogrel, paclitaxel, docetaxel, cabazitaxel, etoposide, teniposide, clevidipine butyrate, remdesivir, carboplatin, valdecoxib, azithromycin, and etocoxib; most preferably, the poorly soluble drug is selected from the group consisting of celecoxib, paclitaxel, docetaxel, ibuprofen, nimodipine, coenzyme Q10, cabazitaxel, etocoxib, posaconazole, cyclosporine, flurbiprofen axetil, dexibuprofen, levosimendan, clevidipine butyrate, clopidogrel, remdesivir, tacrolimus, carboplatin, dexamethasone palmitate, valdecoxib, azithromycin, and propofol.

6. The liquid concentrate according to claim 1, further comprising a pH adjuster, an antioxidant, or the both.

7. The liquid concentrate according to claim 1, wherein
The poorly soluble drug is celecoxib, the phospholipid is egg yolk lecithin, the non-phospholipid emulsifier is polyethylene glycol 15-hydroxystearate, the oil is medium chain triglyceride, the co-emulsifier is absolute ethanol, and when the weight of the poorly soluble drug, the composite emulsifier, the oil and the co-emulsifier is regarded as 100%, celecoxib accounts for 5% by weight, the egg yolk lecithin accounts for 3% by weight, polyethylene glycol 15-hydroxystearate accounts for 48% by weight, the medium chain triglyceride accounts for 28% by weight, and the absolute ethanol accounts for 16% by weight;
or
The poorly soluble drug is ibuprofen, the phospholipid is egg yolk lecithin, the non-phospholipid emulsifier is polyethylene glycol 15-hydroxystearate, the oil is medium chain triglyceride, the co-emulsifier is absolute ethanol, the liquid concentrate further comprises citric acid, and when the weight of the poorly soluble drug, the composite emulsifier, the oil and the co-emulsifier is regarded as 100%, ibuprofen accounts for 12% by weight, the egg yolk lecithin accounts for 2% by weight, polyethylene glycol 15-hydroxystearate accounts for 48% by weight, the medium chain triglyceride accounts for 27% by weight, and the absolute ethanol accounts for 11% by weight;
or
The poorly soluble drug is docetaxel, the phospholipid is egg yolk lecithin, the non-phospholipid emulsifier is polyethylene glycol 15-hydroxystearate, the oil is medium chain triglyceride, the co-emulsifier is absolute ethanol, and when the weight of the poorly soluble drug, the composite emulsifier, the oil and the co-emulsifier is regarded as 100%, docetaxel accounts for 2% by weight, the egg yolk lecithin accounts for 3% by weight, the polyethylene glycol 15-hydroxystearate accounts for 48% by weight, the medium chain triglyceride accounts for 28% by weight, and the absolute ethanol accounts for 19% by weight;
or
The poorly soluble drug is paclitaxel, the phospholipid is egg yolk lecithin, the non-phospholipid emulsifier is polyethylene glycol 15-hydroxystearate, the oil is medium chain triglyceride, the co-emulsifier is absolute ethanol, the liquid concentrate further comprises citric acid, and when the weight of the poorly soluble drug, the composite emulsifier, the oil and the co-emulsifier is regarded as 100%, paclitaxel accounts for 1.5% by weight, the egg yolk lecithin accounts for 3% by weight, the polyethylene glycol 15-hydroxystearate accounts for 49.1% by weight, the medium chain triglyceride accounts for 29.4% by weight, and the absolute ethanol accounts for 17% by weight.

8. Use of the liquid concentrate according to claim 1 in the manufacture of an emulsion, in particular an emulsion for intravenous injection, for example intravenous infusion, wherein the emulsion has an average particle size of between 20 nm and 4000 nm, preferably between 20 nm and 1000 nm, more preferably between 20 nm and 500 nm, and even more preferably between 20 nm and 300 nm.

9. (canceled)

10. A process for preparing the liquid concentrate according to claim 1, comprising the steps of mixing the poorly soluble drug, the phospholipid, the non-phospholipid emulsifier, the medium chain triglyceride and the absolute ethanol in any order, stirring to be uniform, filtering, filling, and capping to seal.

11. An emulsion obtained by diluting the liquid concentrate according to claim 1 with an aqueous vehicle, wherein the aqueous vehicle is an aqueous vehicle suitable for injection selected from the group consisting of water for injection, 5% dextrose injection, and 0.9% sodium chloride injection, and wherein the emulsion is used for intravenous injection, in particular intravenous infusion.

12. (canceled)

13. (canceled)
